SEQUENCE LISTING (27 sequences)

SEQ ID NO: 1 - DNA, 8107 bp, Homo sapiens
  CDS (141)..(4136)
tgagggaagaagaggaagcgggaggagcttggcttcctcgcgtatttgaggacagcccatctcccttcaagaaccctacggagagtcggactgcatctccgcagcgagctcttggagcgccgccggccgggaggcgaaggatgcaggcggctccgcgcgccggctgcggggcagcgctcctgctgtggattgtcagcagctgcctctgcagagcctggacggctccctccacgtcccaaaaatgtgatgagccacttgtctctggactcccccatgtggctttcagcagctcctcctccatctctggtagctattctcccggctatgccaagataaacaagagaggaggtgctgggggatggtctccatcagacagcgaccattatcaatggcttcaggttgactttggcaatcggaagcagatcagtgccattgcaacccaaggaaggtatagcagctcagattgggtgacccaataccggatgctctacagcgacacagggagaaactggaaaccctatcatcaagatgggaatatctgggcatttcccggaaacattaactctgacggtgtggtccggcacgaattacagcatccgattattgcccgctatgtgcgcatagtgcctctggattggaatggagaaggtcgcattggactcagaattgaagtttatggctgttcttactgggctgatgttatcaactttgatggccatgttgtattaccatatagattcagaaacaagaagatgaaaacactgaaagatgtcattgccttgaactttaagacgtctgaaagtgaaggagtaatcctgcacggagaaggacagcaaggagattacattaccttggaactgaaaaaagccaagctggtcctcagtttaaacttaggaagcaaccagcttggccccatatatggccacacatcagtgatgacaggaagtttgctggatgaccaccactggcactctgtggtcattgagcgccaggggcggagcattaacctcactctggacaggagcatgcagcacttccgtaccaatggagagtttgactacctggacttggactatgagataacctttggaggcatccctttctctggcaagcccagctccagcagtagaaagaatttcaaaggctgcatggaaagcatcaactacaatggcgtcaacattactgatcttgccagaaggaagaaattagagccctcaaatgtgggaaatttgagcttttcttgtgtggaaccctatacggtgcctgtctttttcaacgctacaagttacctggaggtgcccggacggcttaaccaggacctgttctcagtcagtttccagtttaggacatggaaccccaatggtctcctggtcttcagtcactttgcggataatttgggcaatgtggagattgacctcactgaaagcaaagtgggtgttcacatcaacatcacacagaccaagatgagccaaatcgatatttcctcaggttctgggttgaatgatggacagtggcacgaggttcgcttcctagccaaggaaaattttgctattctcaccatcgatggagatgaagcatcagcagttcgaactaatagtccccttcaagttaaaactggcgagaagtacttttttggaggttttctgaaccagatgaataactcaagtcactctgtccttcagccttcattccaaggatgcatgcagctcattcaagtggacgatcaacttgtaaatttatacgaagtggcacaaaggaagccgggaagtttcgcgaatgtcagcattgacatgtgtgcgatcatagacagatgtgtgcccaatcactgtgagcatggtggaaagtgctcgcaaacatgggacagcttcaaatgcacttgtgatgagacaggatacagtggggccacctgccacaactctatctacgagccttcctgtgaagcctacaaacacctaggacagacatcaaattattactggatagatcctgatggcagcggacctctggggcctctgaaagtttactgcaacatgacagaggacaaagtgtggaccatagtgtctcatgacttgcagatgcagacgcctgtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccagatggaagcccttacacttggtgggttggcaaagccaacgagaagcactactactggggaggctctgggcctggaatccagaaatgtgcctgcggcatcgaacgcaactgcacagatcccaagtactactgtaactgcgacgcggactacaagcaatggaggaaggatgctggtttcttatcatacaaagatcacctgccagtgagccaagtggtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggctgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttgaggggtggctacttggctatgggatagggaggagggaattactagggaggagagaaagggacaaaagcaccctgcttcatactcttgagcacatccttaaaatatcagcacaagttgggggaggcaggcaatggaatataatggaatattcttgagactgatcacaaaaaaaaaaaaaacctttttaatatttctttatagctgagttttcccttctgtatcaaaacaaaataatacaaaaaatgcttttagagtttaagcaatggttgaaatttgtaggtactatctgtcttattttgtgtgtgtttagaggtgttctaaagacccgtggtaacagggcaagttttctacgtttttaagagcccttagaacgtgggtattttttttcttgagaaaagctaatgcacctacagatggcccccaacattctcttccttttgcttctagtcaaccttaatgggctgttacagaaactagttcgtgtttatatactatttcctttgatgtcctataagtcggaaaagaaaggggcaaagagaacctattatttgccagtttttaagcagagctcaatctatgccagctctctggcatctggggttcctgactgataccagcagttgaaggaagagagtgcatggcacctggtgtgtaacgacacaatcagcacaactggagagaggcattaaagaaccagggaaggtagtttgatttttcattgaattctacaagctaatattgttccacgtatgtagtcttagaccaatagctgtaactatcagctgcaataccatggtgaccagctgttacaaaagattttttcctgttttatctgaaacatactggatttatatatgtataagcgcctcaatggggaattagagccagatgttatgatttgtttgctctttttcttttatagttatagcaaaaatatggataatttctagtgaatgcataaattaggttgcgtttcttattttgctttaaatctctggtagtttttccacccctgtgacacaatcctaatagacagtgtcctgtaaatggacacaacacaataaagtcaagttattattgctgttactctggatgatatggaaaacactgccatattttaaatcaactactccacgtgtttttccatccaatcacactgctgtgattcagggatctttcttctaaaacggacacatttgaacctcaggttcatcacaaacctggtacctgttgcttcccagaggatggagaagtgtagttaatcacacctcttagtttaatctgaaatcttgacccagttatttaacaaataaatacctcattgattatatttaaaagtaatacacttcctgtaaacaaatggggacaatgcatccaaaaaatctttttaaacagattacacaaaaattatttccagaaaggctaccatttatcatcattatatttcaagcctcttatacttaataagcactttctaaaaagtcttgagatcccaccattctgaggaattcaatatgatcactttttccttctttgcctgggagaggttaagaggcggtttcgaaggtatagatgctattgttctgatggcccggctgaataaaatggaaattctagtttgttagaattatgcattctttttcaagattctcagtgtgcctaacttattggagcacatcagtttcttgggtaatggaaaacattacctagagttgccagtggcacattacaccagtacagagcacattccaaaggagacattggaccagttaattcccatacaagtcaaggtaacagaacaaaagggaatcctgatgcccttttaccattgctggttgagctcaggcactgtcatggacacccttaattttaaaaggttttaatcattcttctataaaatacatttaaaatggaaaaatacttaatatcactaaatatcagaacaatgtaacatttacaaatgacatattgaaagcaaaggctgttttatttagccaagatgattaccattaggagttactttatgtattgttgaaagcaaattttaaacatgatgttttagaagtgtttctgatttttaaacctggtttacaggtattacttctgcacttaccaaataatgccagatggaaatttattatttcttgcaattcccgtgatagctctgttctttatgcattgtctcaacactttcccttttttcccaaaatgagtagagaattaaagccacccaaaacagcttctgctactaaaatgttctcatcctttctcctccctctccttttcctgccacaaaaggtgaaaaatgagatccaatcctctcaccaaaatttcaaacctaggacactggaatgactgcagggatcagtggttctcccatatcaccatcaattaagacatataggacactgtcttccttcaagagggttacaatgtggccatcagacaggaaaccaaacggtggataaagtattaagtaactaagtgccaaataaatgctggaaatcttgacctctccttgggattatgggtgtaacaaaaatccctacatctgtttatgaaggccatattcagtacattttaaatggtaaataatctgtttatgtgaagaaaaagaattaagtctttcttccaactctctccttggatagcctagcacagtgcagcctccataaccatgacattcccgcccaagctctcagtgcctaatcctgctttgtcattcacatctcacaaaatcttgacatcttacattccaatacattatcaagcaagcacaagtatgctggtagtagcctctttaaataatatgtatagacaacaacaacgacaaaaaatagactgttttaaagtttcagggaaagttggtggctgatttaaagttgtgcaggaaacatcttctgtgtatgaagcaaatgtcgatgttttgaaaagctaggagatgactttgaatgaatgcaaggttagtgagatcctaagctctcaaaatagcatattccctagagctcaagaaagctggtccaggaggttgaaaaagctattttgttgttaaattattttctggcccttcttaatatttaaaaatgtatttccccttgtggctttcaaccacctgctcaaaaaaagagacttgttacatgaaagttttcattaaagagctgaaaacaagaatttagagagccattcctagaaaatgtcctactgccctgcatttgacaaacaagcatcctttactaacaagagcaggaattcagaggcacaagaaaaagcattggcatgagccaaagagtctgtcttaatgttacttttgaaaatctgctgagcggccaccatatgcaggctgagagctgggcacaggcgaagccattggaagcacttcaggaacaagcacacagctgtgggacttgaacatgcaagtgttcaggttgtgtcaagaagcttttctttccttctatgatggaatctgttcttttctatcctacttttttctctcttcctctcctcaccacattataccctgctcttacgcagtaaacgttttaatggcccgtttatgtctcatgcctccaaacaacactgaatttgaaaccccccattttttcttttcaccaccctgttgagcaattttcccaaaaaaagggcagcaattattaaattgaattcaagtttctagattttactaagttttattttgtcaggttttttaaattttttcagtgagcgtggtgactgcagaggttagtgctgtgaaaagctgggctaaatattctttctgtaaagtcaaacaggattccatcccctgtgaaataacacaaaatttcactctctaaaagcaacagcatgtaaactagaatgaaagaaggaaattatgtacgtatgcctaatattctttgtgaatgtctttcatttaactaaaattatattagaaaccagattgataaataaaaaattcaaagtagttttaattatcct

SEQ ID NO: 2 - protein, 1331 aa, Homo sapiens
MQAAPRAGCGAALLLWIVSSCLCRAWTAPSTSQKCDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGCSYWADVINFDGHVVLPYRFRNKKMKTLKDVIALNFKTSESEGVILHGEGQQGDYITLELKKAKLVLSLNLGSNQLGPIYGHTSVMTGSLLDDHHWHSVVIERQGRSINLTLDRSMQHFRTNGEFDYLDLDYEITFGGIPFSGKPSSSSRKNFKGCMESINYNGVNITDLARRKKLEPSNVGNLSFSCVEPYTVPVFFNATSYLEVPGRLNQDLFSVSFQFRTWNPNGLLVFSHFADNLGNVEIDLTESKVGVHINITQTKMSQIDISSGSGLNDGQWHEVRFLAKENFAILTIDGDEASAVRTNSPLQVKTGEKYFFGGFLNQMNNSSHSVLQPSFQGCMQLIQVDDQLVNLYEVAQRKPGSFANVSIDMCAIIDRCVPNHCEHGGKCSQTWDSFKCTCDETGYSGATCHNSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 3 - DNA, 401 bp, Homo sapiens
  allele (201)..(201): A/G
tgaagactagagtcagggacggtttcagagggaagatgggtcattagctggatctcaggtatttgagatgatcacatgtatttctcaacccgtctcctctggagaagtggaatttttggtccatttcatttctggtatgtctatttctttattaaccattttcaaatttcctcttttgtctgtgactttcaacagcccaartctgtctctatctcaagtcctcccacgccacccccctccaagtccctgtctgtgttccaatcccctgcctctcctaacctctcttcacactcttctcttccaaagactccccccaggtacagtgccttcgagcctacaagccccgagagaatgatgaattggcactggagaaagccgacgtggtgatggtgactcagcag

SEQ ID NO: 4 - DNA, 604 bp, Homo sapiens
  allele (478)..(478): C/T
tcctataagcatcccttggtaaccaatattattttctttaccaaaattcagagagaaaaatgaacaaatgttatggagcccaaactatcagagtcagaagaggccccttttccttcaaacaacacctatgaagttatacacactgctttgggggaaatcaatcaattacaatgctcacttcccactagagggagcctcctatctttctgaggaaatcttgaaaaggcaatttaaaaattcaggaacagtactgcaagataatctattgtcagtattaccagttcacacagcttatttattaaaactcaaattatttttcatagttctttatgtttttactagtgcaaacacaaaagtaaaactacacagtaatacagagtgaatctcatgtgaatattgccctgatacgaacttcaatataggaagctcttaactcttgagtcatttgggacttgggacttctgtgagatgatgtttytgaacactggtgatatacacaaaaatatgtatcgatctagacactgttacatagttggtagatcagggtggtgatctttgagggattcacagcaaagtcaattgaaatctctacctctcctgaata

SEQ ID NO: 5 - DNA, 436 bp, Homo sapiens
  allele (236)..(236): A/G
tgctgtcagcaccatcaggagaatttatcaccacacaaataataataataataataataataataataataaatgtgtcactgcaattcctactagaaactcaactgccatcagagtgcacaaagtcccaaatttatataaggaacagctgggtcacttccatgacattcaagcttgggctggagtagcgacgttttgtctaagccaactcagaaccctttataatactcctcccrcctataattttctgttttaggctctctgaaatatcctccctttctgttacctttacctgaccattacctcgaggaatctctcaaggctcacactcggaatcttttttactgcagcttgtagtccacagtagctggctctttactttgggcacagctattgatctctgcccggaagtatggacttgatcttgggacttccc

SEQ ID NO: 6 - DNA, 628 bp, Homo sapiens
  allele (247)..(247): C/T
agcttcactcaaacataaattatgatttagtatgtcttgaaggtatcattttgcctgacagtataagcaaaaagcagaaaatagtgattgattaaccttattttgtagtgatgcaggaatctaataaacccaaattaaggattgaaatgaagataacaaaattgaagtaaccaatatttctggttggagcccacttttattacagcgctaaaaatagtcattgacataagaaaggatagatgaatgycatcttgttattttccagcactgagataggtttagatgagtatgtcggagataggaacattaagaagttggtagccctcgcacatatgatgaataaagatagatcattataaatggcttcaaaactcttttcttcgaaggttcttactgggcttattataaaaggaacaaagattgttagaagatatgctgtcttcactgtctcaaaaacaatactttttgaaaccttctcaagaatatagtgaaatggataaagaacctctctgagaaactcctgttgtcattggattttgaaagttgtcataaaccacctctgtacctaacctaaataatgaatgttgttgtctttgttgtatttaaaatataagtattgctaccaagt

SEQ ID NO: 7 - DNA, 466 bp, Homo sapiens
  allele (201)..(201): A/G
gtaactataattgtactattttcctgtttttttttcttttgtcttctttggctattttgatgccttttaatgcatccccctgttgatgtacagtttaatatctgatataacactaagattaattttttaattgttaatatatctttgactagtctttccctatattattaccattgtgattacttttacttactacgatcraagtagcaacttgtagtcagatagcagtgatattcacggtctatcaatttatccattcatccatttatccatcctacttttcttcccttcccccatccatccattatgtactgtggatttgcactgaagtcaattggaattaagtattataaaactctattttactaatagccatataaaacccttaagaggcaattcagacattcacaatacttggaattttaacagttacctctattgactattacaatatagcataatctgt

SEQ ID NO: 8 - DNA, 401 bp, Homo sapiens
  allele (201)..(201): C/T
aacatcttgaatgtagtgttagttatgatgatatataccgtttctaaaatttctccatatgcacattgaaaatgaatacattttattacatgtaaattcttaagagatgtaattttaaaaaagagctactgaaatgctaaacaagacagaaaaatgtccctcgagattcttgctattgagctgacgaccaaattcaggcayttgcagagtagttgaccttctcttaagaaggagccctcctctgtgccctcactataagaaggagcctcccaacattagattttctcttgaagagccctttggactgttgttctgtctgagagctactggagaatctctttgatagctttgcttttgacggttttctctggagaaaggtctaatagaagcaaacagttaaa

SEQ ID NO: 9 - DNA, 717 bp, Homo sapiens
  allele (466)..(466): A/G
tcaggttgggagagaaatgatattgaagggacaaaatagcaaacataagaagccacctttgctcatttctgcttcccagcataatttcccaaagcgcttggccctgtgacaacatacagtgctccaaaaaaaaaaaaaaaaaaagaggtcgaaacaaaataaaacataccccactacgtctcttgcatgagtcagtattccttaaaaaataaatgaccctaatcattgcttttttctaacataaaataatgtctgataggattaaaaattatgcctctataatctgtaatcatgaatcatataatctatcagtatcatataatctataagatcatatcttatcatataatctatatgagtgtagcttatagattacatcttatagataagatatactcttgtccctcattaccttaggggaagctctcgttcactggcagattgccatggctaattccagctgccrttcattcaaatgttaatacggatataggattttatttatttatggtagagttgtaggaaaacaaaatgtctctattaacatttctctattttgctaaaatattctgaaattataggctctgataaataatatgttatatcttataggtaagatacactgttacatccaaactttaatataattttacaggtactaaccccccattacctatatgtaaacatcagaatcaaaaattgatcccttgacttaac

SEQ ID NO: 10 - DNA, 401 bp, Homo sapiens
  allele (201)..(201): C/T
caatatatgaggagataaaatgaattttccacaaactatgattctgtgtcatctaaacattagcttgcacaggccttttaaagaaagcagagtgaggactccctggccagtgttctctaccatctcttctgcctaccttcttttctctcatggaagtaagaaaagaatccatttcatcaaaggttgaacattccacttcayccctgaattctctcttgctttgagttcttaggtacatctatattagatatcactttctcctctgcatccccaatgcccttttccctcctcagcatacctgatcctctgtccttgctgacctttgtatgtgtgtgttttctcccttgatgacatatccctcttcagctattgctctatttatattcagaatcccaggcaag

SEQ ID NO: 11 - DNA, 692 bp, Homo sapiens
  allele (211)..(211): A/G
agatagatgatagatagatagatagatagatagatagatagatagatagaggagagagagagagagagagagattagatggataggtatgtagtttcacctataagcaaaggcaaaaatgagtatctcagaaaggcagatttagggaccactaagattttaagttgcatcattctgtaaattggagaatattatcacttactgtactgtcrcaaatttcagataagttaacattcctgaatgttcttggcacagcataaatgctcaatatcttcaattatcatactttttccccttcatccctgccctgacgcatgatagatcatttcttaaaatatactacagcatgtattaaataatatttctactgctcagttttctggggaaaatagtgtttagttaatttctaggttttgttaagaatttacaaaatatatttcataaatgtgttcttttcctttcagaaataaatgtataattttactcacaacatttcaacaactttcctaacttgcatacttcatttttctagtgtttcataaaagttctaaatttaatcatatgatattttaaaataagatattaagcaagaaaacacagcatctattgtatttttaaacgaatcttatggtttttattttattatttcaattactgttactaaattattaattctaggttagaattataaag

SEQ ID NO: 12 - DNA, 401 bp, Homo sapiens
  allele (201)..(201): A/G
tgtttgaaatattagaagtatttcataaggctcctatatctgtttttctagactacatattgagaactgctgtaattacagtatcatcctcagtgttagatgtaaaatgttaagaaaaatctgtccatctggcaagaggaggtaaaagtttgttagagtgggcagaagttaggtctgatagcacctaggtctagagaaacrcaaactccaggtaatacatatattctagtaaggtttgctccatttaaaaaatacaaacttatagaaacagaaaggcatttattatgctcatttagtagcaataaatgccttctaatatctttgaaaatttatcatgcattctcttcttaaatgtcttcaatgtgtacagagcccatagttaataacaattctaattatta

SEQ ID NO: 13 - DNA, 401 bp, Homo sapiens
  allele (201)..(201): A/C
ctgttttaaattcataaaactgttataaatttatgtgatatagctaatatgaaagacacctgaattaaacactttaatgaaatgaatatttattatcagacagaggtttagaataagaaatgtcaatatatagaactgcctccactaaacctttttcctgatgaatttaagagatgtgtttattgaacagaagcacaattmatagataactcatctttatagggcgtctgttgccatggtaatagtgatgtgacatcgttcaaagggaagtgtttgttctaaaagcaatgatgagtaacatcctgcagcctccattcaaaactaaaggtaggtaggttgggctgttttcctaggcacagacaagggtgtagaatcctgacaatctctctgaagtacacccc

SEQ ID NO: 14 - DNA, 628 bp, Homo sapiens
  allele (428)..(428): A/G
tagtttacaaaatatgtttacatatatcattgaatttgatactcctaagaaccccataagcaaagtatgatctcttattatacatattctatagataagaaactgagcatctagaagttaagtgactgctcaagatcacgtggctaggagtttacccaaagcctaaacttaggtcttctgatttctaatctagtgtttctcctactataatatttcagtctgctttttcctttcactctaaggcttattattttcactaaattcaaattgccttttctgctttgttaaatactagagttagacatttgtaaagttaaacacacacacacatacacacacacacacacgacattaagggcaagatatatacttccaaggtttctgagaggtacagcttatacaaggaaaatcttttgtaagaaattgtrtagatctacttattcatgtgctgagtacacattttcaattttgactattggtataatttagagaaattcaattaaaaagaaaaaaataagtactcctctttaggcatttattctgttcacctgaaagtatattcatagccattttttttttgttaatgttaaataacagatccctttccagggatcctttccagggatcc

SEQ ID NO: 15 - DNA, 663 bp, Homo sapiens
  misc_feature (137)..(137): N = A, T, C or G
gctaaatgttgcataatacatgtgggatataagggatccaactttaaggacttaaaaaaggctaaaggtgttatatatctaaaaaacattctctgtattttatgaactggagrtaagagtcatagcagaaacttccnaccattagtctccttggcaatactgattaacctccctcagtcacaattttgcacaaaaagaatagaaatgttaaagaaatgctttgggatttcaaggctatcattaacatttatgggaagagaggtgaaagagctagctagtaattatcaatgtactgcattgctctacatctctggcaggaacccaactttttggatcaactagcaggcagcaccgttagccatgagcttccatggtcacgatgagattgtatgtcttggcatgattcggcaggtgtcatttatatgcaatgtcatctgcaggaaaaatgcagggagtatcattctccctgcttaagttcttttatggagacaactcaaagtcttcgctaaatgattactcttgatcattttcagttacaacaatgacttaagagtattgtggaaaagcatcccagtgaacagaactgttaagatgaataaaaatgtctaaattctaaatatatattttcaactatttgcaaggctatatgaacatttttgaaat

SEQ ID NO: 16 - DNA, 3389 bp, Homo sapiens
  allele (3189)..(3189): A/C
ttggtgaagctatacttactgttttttaaagaaaaaagcttatcataacttatttaaaagacaaattttgttttgttttgttttggttttttatttttttattttttatttttattttttaattttattattatactttaagttttagggtacatgtgcacaatgtgcaggttagttacatatgtatacatgtgccatgctggtgcactgcacccattaactcatcatttagcattaggtatatctcctaaagctatccctcccccctccccccaccccacaacagtccacagagtgtgatgttccccttcctgtgtccatgtgttctcattcttcaattcccacctatgagtgagaatatgcagtgtttggttttttgttcttgcgatagtttactgagaatgatgatttccaatttcatccatgtccctacaaaggacatgaactcatcatttcttatggctgcatggtattccatggtgtatacgtgccacattttcttcatccagtctatcattgttggacatttgggttggttccaagtctttgctattgtgaatagtgccgcaataaacatacgtgtgcatgtgtctttataacagcatgatttatagtcctttgggtatatacccagtaatgggatggctgggtcaaatggtatttctagttctagatctctgaggaattgccacactgacttccacaatggttgaactagtttgcagtcccaccaacagtgtaaaagtgttcctatttctccacatcctctccagcacctgttgtttcctgactttttaatgattgccattctaactggtgtgagatggtatctcgttgtggttttgatttgcatttctctgatggccagtgatgaagagcattgtttcatgtgttttttggctgcataaatgtcttcttttgagaagtgtctgttcatgtcctttgcccactttttgatggggttttttgtttttttcttgtaaatttgtttgagttcattgtagattctggatattagccctttgtcagatgagtaggttgggaaaattttctcccattttgtaggttgcctgttcactctgatggtagtttcttttgctgtgcagaagctctttagtttaattagatcccatttgtcaattttggcttttgttgccattgcttttggtgttttagacatgaagtccttgcccatgcctatgtcctgaatggtaatgcctaggttttcttctagggtttttatggttttaggtctaacgtttaagtcttgaatccatcttgaattaatttttgtataaggtgtaaggaagggatccagtttcagctttctacatatggctagccagttttcccagcaccatttattaaatagggaatcctttccccattgtttgtttttctcaggtttgtcaaagatcagatagttgtagatatgcagcgttatttctgagggctgtgttctgttccattgatctatatctctgttttggtaccagtaccatgctgttttggttactgtagccttgtagtatagtttgaagtcaggtagcgtgatgcctccagctttgttcttttggcttagggttgacttggcgatgcaggctctttttttggttccatatgaactttaaaatagttttttccaattctgtgaagaaagtcattggtagcttgatggggatggcattgaatctataaattaccttgggcagtatggccattttcatgatattgattcttcctacccatgagtatggaatgttcttccatttgtttgtatcctcttttatttccttgagcagtggtttgtagttctccttgaagaggtccttcacgtctcttgtaagttggattcctaggtattttattctctttgaagcaattgtgaatgggagttcactcatgatttagctctctgttggtctgttattggtgtataagaaagcttgtgatttttgtagattgattttgtatcctgagactttgctgaagttgcttatcagtttaaggagattttgggctgaaacaatggggttttctagatatacaatcatgtcgtctgcaaacagggacaattcgacttcctcttttcctaattgaataccctttatttccttctcctgcctaattgccctggccagaacactatgttgaataggagtggtgagagagggcatccctgtcttgtgcccgttttcaaagggaatgcttccagtttttgcccattcagtatgatattggctgtgggtttgtcatagacagctcttattattttgagatatgtcccatcaatacctaatttatggagagtttttagcatgaagggttgttgaattttgtcaaaggcctgttctgcatctattgagataatcatgtggtttttgtctttggttctgtttatatgctggattacatttattgatttgcgtatgttgaaccagccttgcatcccagggatgaagccctcttgatcatggtggataaggtttttgatgtgctgctggattcggtttgccagtattttattgaggatttttgcatcaatattcatcaaggatattggtctaaaattcgcaaatagacgcaataaaaaatgataaaggagatatcaccaccgatcccacagaaatacaaactaccatcagagaatactacaaacacctctacacaaataaactagaaaatctagaagaaatggataaattcctcgacacatacaccctcccaagactaaaccaggaagaagttgactctctgaatagaccaagaacaggctctgaaattgtggcaataatcagtagcttaccaaccaaaacgagtccaggaccagatggattcacagccgaattctaccagaggtacaaggaggaactggtaccattccttctgaaactattccagtcagtagaaaaagagggaatcctccctcagtcattttatgaggccagcatcatcctgataccaaagccgggcagagacacaaccaaaaaagagaattttagagcagaaatttttttaaaaagaaattcaaaaaaatcatttattaaaataggcaagtacttctaaaagcagggcttgaaggatgcactgatatgtactttgataatgtaagccccaaagatcaggacaggtgtctgtcttatttgctcttctatctcatttacaattccctgaatgaataatgaatgmatgtgtgtatcacaagaaaaaaatcatggctatggttgccattcgaaagaggcacagagttataacactagtcataggtagtgcagatagaagcaagatcattgcatgggggctggagggaacttaaataactagtagattacatataataacctcgattctcatccctcattctatgtcaggcttagagggtctgcata

SEQ ID NO: 17 - DNA, 401 bp, Homo sapiens
  allele (201)..(201): A/C
gttaacttataatctctcacgcaatggtttctcctagtgtttaataaaatggtattaagggtaactgctactattttttctaaaattagaaggtaacattgaatggacacacagcagatacatgttacattctgaaatgtatgctttgctggagcagaatgaatgcaatcaatgtaatggaaatgctgcactaatggctcmttaaacatggaaagtatgcatagatgaaaaaagtctgtgcatatgtttcagttcactgaaggaagcaatgaactataatgtggaaagaaaaggaaacatacttctttacgtaaatattcttacttctggaaaagaattgttgaatcagtcaggaatttgacagcaagatttcaactattggagcagacaactttatattg

SEQ ID NO: 18 - DNA, 201 bp, Homo sapiens
  allele (100)..(100): G/C
tctatacccaacctgtgaagaaggttcatggcattctagttactatttcaaagggaggactctaaacatttccacccactgcactttgggtgcacaaatstatagtgagacagaatactttcacacaacacattccatgaagcagatttattacagataggcagcaagggacaacagaagcctaggactcatgacaaagct

SEQ ID NO: 19 - DNA, 1679 bp, Homo sapiens
  allele (1373)..(1373): C/G
actgaaaaatctactgaatcaggcttttgtaggtctaatttcagccccttctcttcagactagttttgttgctgttcccagcacaaataaatgaatgtagttaatccaaaaaatatttattgtgtacctactaatgttctggacactattttagatggggagacaattataaacataatagacaggtaaaagtccctgaactcatgtaattaacattctaattgcgggagacaggtgatatagcacacaaataagcaaaagatgcagtatattagaatataatgggctgcatacagtggttcacgcctgtaatcccagcactttgggaggccgaggtgggcagatcacgaggtcaagagatcgagaccatcctggccaacatggtaaaaccccatctccactaaaaatacaaaaattagctgggcatggtatcacaagcctgtagtcccagctacttgggaggctgaggcaggaggatcgcttgaacccaggaggtggaggttgcagtgagccgagatcacaccactgcactccagcctgggcgacagagcgagacttgtctcaaaacaaacaaacaaaaaaagaatataatgaactatgaagaaaaataaagcagataagggagataaggaaaacagaagtgaggttactattttaaatatggttgttaaggtgggtcttcaagagaagatgacatctgggcaatgactggatggagttgatggatcttgccatgcagtaggtaagttggaagaacagcacacgccatgccctgagaatctaatgcttgcccgatgtgttcatgggacagcaacacatccagtatgattaaaatgtgtgagcaactgagtgagcaaacaaggggaagggcagtagaagccaagatcatcgaggtatagttcagggccttggaggctgttgtttttaacatgaggttggacatgaccagaatgcatattttaacagaatcacactggctgctgtgatgagagtgaatagggcctgggtgaaagtaggtcgactaaggcaggaagctactgcaaaaacagacagaggacgatggtgtttgaaccagaatggcggtgatagagtcattgagagcagaactagttgatggggtagacgtgggttgtgagtgtagagaggcagccaggatgggttttggcctcagcaatgggtaggatggcccattctcttctctatactcactgtggataagttcttagtgaattttatgatcttctcatttccaacagcaaaacaccttattattttttttttttagttcaaatctcactttctaagaatcttaacctaattaacctcatgcttttctaaacagtccattaaacttcattcccttgggcatgtgsctaggtttattctgttactttgtcctcattagaatagttttcaggctcttatgttaaaaagcttgaaaaacaagaaacaaatatataatagacttttaaagtagatggtaatatttaaatattgaaacctatttaaaatgcatacacatttaaatatataaaatacatgattttacttatttataaacagaaaaaaaattaaaatagatgtaaaaatctatctacatctattgatttataatatccatatctatatatattttataccttcatctatccaaaaaactgtgatctaattaaacagct

SEQ ID NO: 20 - DNA, 606 bp, Homo sapiens
  allele (501)..(501): A/G
ttgccctgtaagtattatgctttcaaggaagctctaacaactggttatcgctgaggttagtgtctctcatggtactcgcttcatgctctgtccattaggtaaatgacatgcaaagatggcttcctctaagtttctgagacacaggtactcatatttaacttccgtggaggaggaacacaggagtgaaatagaaaggtctgcatttggtgtctgtcttcccatccattcctaagagaagatgacataggcctactgcttatcaagagcatttgaccctaatgagtgtcgttatagaaaagttagaacctttctataagactttgtattttactttccagaaaattccaattcactaaactgtccatttcttctcctactagtgtgttattttcagggtgttttcttcctattatagtctcagaaggcccatgctcagcttccgtagaccctgtcctctgtcagtcagtcacttcatttacccctaggttttgactcttcacrgatttctggatatctccctccaaagggatagctcttctgttgctaatgactagcatgagggttggtaagtagtgacagtaaaattaacattccttgaccacatga

SEQ ID NO: 21 - DNA, 401 bp, Homo sapiens
  allele (201)..(201): A/C
ttacatgttcctggtttaatcataagacaaatggcctctctcttctggtaacaattttatattcctggtcaagggagctaggttggcccacacatgagagggaacagtggaagtaggatgtgtaatgcccagaagaacaggcatcggaggagagcccaggagttgattctatatgtaaatctgttataataccaatccgamcaagctagtattctatcaaatagccaaatgcagaggtaagcaggaagtaagtaatatcttttgtgattttttaaaatgacacacagtagatgagctcagaacatatacagtacagtagccactgggcaaccattagataaacaagaaaagagccagaccatggggatgaaaccatctttaatcatggcaagtggaataat

SEQ ID NO: 22 - DNA, 605 bp, Homo sapiens
  allele (307)..(307): C/T
caatatgatgttctgacagcacagtaggaagcaatgtcaagtgaactaaacacaacctttcatggaaattgttgcttagaatgagaataagattttaaaaagggagtccattaaaaacatatttattaaataattaaataggtggtctggggcttggtgaaacttctgaagacaatgcacaaatcactaaagtttgagaattgctttcttaaggtaattaactatctatcagaaattggctataaatggcatattaataatgtaaacttttctcatttttcaataattcaatgtctctgacagcaayaactgtatcactggcatattataacagattatttgaaataattttatacatacttataacattataagttccacacaatcagattttatgtgtgtttgtcactgtgtacctatgcctgaaacataagatgcactcataaatattggttaagtaaatacattaacaattatgcaagcttatttaaacacattaaaattctaaaaactgtacctgggagataacaaagaatgataaacatagatatttcataagtctgggaaacgattcacaccctctctctgctatctgataaaacccc

SEQ ID NO: 23 - DNA, 401 bp, Homo sapiens
  allele (201)..(201): C/T
ctgctaaccgatgaccctatccccaaagctgtttccattcattctctttgttgagagtatgtcaaaccatcccaggatggaggctggagatgagtttaaccccacctgcaactgttctctcgatggagctactctacagaaaaatcatgttccattagttcactaaatcataacaaaagtaaatgtacttgtttgaacatygtacaaactagtcctaaaagggttctcacgtattttatatgcgcagtctttagggaggcaaaaatgttttatgcaatctatacaagaaagttatattttcagctgttatccaaaacaaaggaagcgtactctgtgaaaaattggtattattgtttaacaatatccatttccagaggaatgcttttcttaagtagaagttg

SEQ ID NO: 24 - DNA, 844 bp, Homo sapiens
  allele (344)..(344): C/T
tgagtgaaaggcgtcaggaaagccatttgtaaaatgatgatgaggacgtgcttcttgcctgcaggttaatcccaaggtgatcacaaatagctgcacgatgatgccaggcacaatttgatgtatgggaggcagcaagcagggatgaagaaagcatgaactgtggtcacaaagatctacttctatgtgttggcttcgccgctcatggcatgtgagcttcccaggtcctatttgggcctcagtctccttatgtgtggcattggcataacaatatgcaccgcgtaggagtgtgttaataacagataatgcacttctttgcactaattctagttactggtagctgttaygattattgctattattgagtataggcacattctatagaagcacatagttggaataaattaaatatggctgagatgatgtgtatataaatctgatactagaggtatcaggagtgctatggactgaactgtgtcccctcaaaatttgtctgttgaatccctaacctccaatgtgactgcatttgaagatgggacctttaaagaagtacttaacattaaatgaggccataaaggtgagacctaatccaatagaactggtgttcttgtaagaagagggacagacactggggatgctcttgcacagagaaaagatcaagtgaagacacggcaagaagacagcagtctgaaaaccaagaagagaagcctcagaagaaagcaaccctacggacacactgatcttggacttccagcttttagaactgtgagaatgtttattgtttaagccaccagactatggtattctgttagggcagcccaagcagaccaagacaatgaggaagggt

SEQ ID NO: 25 - DNA, 701 bp, Homo sapiens
  allele (501)..(501): C/T
tacaggtttcgggtttcacagaaacaacagcaaacccaatagtgcttcactcagtttccctcccaaagacagcaaagagagaaaattaactacctggccaggcgcagtggctcacgcctgtaatcccagcactttgggaggccgaggctggaggatcacctgaggtcaggagtttgagaccagcctggccaacatggtgaaaccccgtctctactaaaaatacaaaaattagccaggcatggtggtgggtgcctgtaatcccagctacttgggaggctgaggcaggagaatcgcctgaacccgggaagtggagattacagtgagctgagattgcaccactacacttcagcctgggtgacagagtgagactccgtctcaaaaaaaaaaaaaagagaaaacaaaaaaccaactatctaaggcagaatggtaataaactgcccagtaatgctttcagggataattaaatgagtgatgagggcacaattacaaataagctaaccygagttacttggaaagcagatggaagcacccaagaccatacaacaagaatgaaaccccagaacagtaaacacacggtattcctaccatatatgccccccatcggtgtcacctaaaccaacatctctttgaccttttttttactcatgtgaccagttatttgattgtttggctgtcaaagtcacaccaagataataaattag

SEQ ID NO: 26 - DNA, 953 bp, Homo sapiens
  allele (453)..(453): A/G
acatgtaccggatcaggaagaccagggtgcacaggatggtgaaaatcaccacagcaatgacgcctgacggagaaaggagaagcacgagaggttagacagggcttgggagtccctccaacactgtccccacttcactacagctccgtcagacacagccctctcccatataaaacacaacctccaaaacaacaaagataacaaaaatgaagtaagatgtctgtttttgtccatgaaggagtaacaggaaaaacaggagtaacttgtcttccagccaaaagtaactaaaatactggacacagtctatgaaatgacagtctacagacattagaaaacaggcagaaaggaaccatgatgtctgagaacagagaaacaaaaaggaggaatcctacatttttcctagttttctggctggcagcgatttttggacagcagtgtagggaggggcagcacaaraaagcacagtggtttccctgaatggagaggggagatagagtgggcgcgcgagagagctatgtatagaaagaaccctaataatctgcacataagtctctctgaatttgtggccgaataatactaacatgtgcatgtacagaataaaagtccacaggccaggcaaaaaaccagagctcacggagggctgggagatgtttatagtccaacaaccacactgaagagacctcattgaacaatcagagcatttaatacagttgatagaatggtcatgctgaaggtgtttgctaaaataggcctagaataaagactactctagctctgtcctgaaaaagctttaaaacacatctaaataacgtcaagctgcagagaaaaataattcagagaaaaaaaatgtagcaaaatgtcacttgaccagaagaaacatcagtcaattagaaggagagccagaaaggacagagcatggaatgagcagatgaggacagtacaatagctattagaaa

SEQ ID NO: 27 - DNA, 718 bp, Homo sapiens
  allele (518)..(518): C/G
gatttaataaatctgccactgaatcaaaatacaggagccttgtcaccttgaagttgcatacatgtgttgtccaatacggtggccgccagccacacatggctattgaccacttgagatgtggcttgtctgaactgagatatgctttaaacataaaatacacactgcattttaaagacttagcatgaaaacgaagaatgtcaaatagctcattaagtaatttttacatgttgtttacatgttacaaagatattttcaacatactgagaaatgaaacgttgttaaaattgatcttacctgtctgtttttactttttaatcttgctactagaaaatttaaaattgcgtatgtggttcacatcatacttttattagaccacactactctaggaagagaaataatcactcattttcttgtattacttctcattttcgtccatatcaattcaagacgttagatttcctcctgcctgtcacagattctattagatttaaaaccacagggtcttttgctcttttctstaacttctaccttcaatatgcatttctcataaaatgtatctttgcattttaaattcaataagcacatcaatatcaaaactgcttttattctcttcacttatcaccccttcctattcaggacattactctttcttttctccatcacaatttattttttcctgacatttttttctggcttccttttcaaattagaaacctgc

(19) United States
(12) Patent Application Publication
(10) Pub. No.: US 2009/0208482 A1
(43) Pub. Date: Aug. 20, 2009

(54) HUMAN OBESITY SUSCEPTIBILITY GENE ENCODING A MEMBER OF THE NEUREXIN FAMILY AND USES THEREOF

(76) Inventors: Anne Philippi, St. Fargeau Ponthierry (FR); Francis Rousseau, Savigny sur Orge (FR); Elke Roschmann, Corbeil Essonnes (FR)

Correspondence Address: OCCHIUTI ROHLICEK & TSAO, LLP, 10 FAWCETT STREET, CAMBRIDGE, MA, 02138, US

(21) Appl. No.: 11/719,831

(22) PCT Filed: Nov. 21, 2005
(86) PCT No.: PCT/IB05/03337

§ 371 Date: Mar. 26, 2009

Related U.S. Application Data
(60) Provisional application No. 60/629,946, filed on Nov. 23, 2004.

(51) Int. Cl. A61K 39/395; C12Q 1/68; A61K 38/00; A61K 31/7088

(52) U.S. Cl. 424/130.1; 435/6; 514/2

(57) ABSTRACT

The present invention more particularly discloses the identification of a human obesity susceptibility gene, which can be used for the diagnosis, prevention and treatment of obesity and associated disorders, as well as for the screening of therapeutically active drugs. The invention more specifically discloses certain alleles of the contactin associated protein-like 2 (CNTNAP2) gene related to susceptibility to obesity and representing novel targets for therapeutic intervention. The present invention relates to particular mutations in the CNTNAP2 gene and expression products, as well as to diagnostic tools and kits based on these mutations. The invention can be used in the diagnosis of predisposition to, detection, prevention and/or treatment of coronary heart disease and metabolic disorders, including but not limited to hypoalphalipoproteinemia, familial combined hyperlipidemia, insulin resistant syndrome X or multiple metabolic disorder, coronary artery disease, diabetes and associated complications and dyslipidemia.

### FIELD OF THE INVENTION

The present invention relates generally to the fields of genetics and medicine.

### BACKGROUND OF THE INVENTION

Approximately three to eight percent of the total health costs of modern industrialized countries are currently due to the direct costs of obesity (Wolf, 1996). In Germany, the total costs (both direct and indirect) related to obesity and comorbid disorders were estimated at 21 billion German marks (29.4 US Dollar) in 1995 (Schneider, 1996). By 2030 these costs will rise by 50% even if the prevalence of obesity does not increase further.

Obesity is often defined simply as a condition of abnormal or excessive fat accumulation in adipose tissue, to the extent that health may be impaired. The underlying disease is the process of undesirable positive energy balance and weight gain. An abdominal fat distribution is associated with higher health risks than a gynoid fat distribution.

The body mass index (BMI; kg/m²) provides the most useful, albeit crude, population-level measure of obesity. It can be used to estimate the prevalence of obesity within a population and the risks associated with it. However, BMI does not account for body compositon or body fat distribution (WHO, 1998).

TABLE 1
Classification of overweight in adults
according to BMI (WHO, 1998)

| Classification | BMI (kg/m²) | Risk of co-morbidities |
|---|---|---|
| Underweight | <18.5 | Low (but risks of other |
| | | clinical problems increased) |
| Normal range | 18.5-24.9 | Average |
| Overweight | ≧25 | |
| Pre-obese | 25-29.9 | Increased |
| Obese class I | 30-34.9 | Moderate |
| Obese class II | 35-39.9 | Severe |
| Obese class III | ≧40 | Very severe |

Obesity has also been defined using the 85^{th} and 95^{th} BMI-percentiles as cutoffs for definition of obesity and severe obesity. BMI-percentiles have been calculated within several populations; centiles for the German population based on the German National Nutrition Survey have been available since 1994 (Hebebrand et al., 1994, 1996). Because the WHO classification of the different weight classes can only be applied to adults, it has become customary to refer to BMI-percentiles for the definition of obesity in children and adolescents.

The recent rise in the prevalence of obesity is an issue of major concern for the health systems of several countries. According to reports of the Center of Disease Control and Prevention (CDC) there has been a dramatic increase in obesity in the United States during the past 20 years. In 1985 only a few states were participating in CDC's Behavioral Risk Factor Surveillance System (BRFSS) and providing obesity data. In 1991, four states were reporting obesity prevalence rates of 15-19 percent and no states reported rates at or above 20 percent. In 2002, 20 states have obesity prevalence rates of 15-19 percent; 29 states have rates of 20-24 percent; and one state reports a rate over 25 percent. Similar trends have been observed in other countries in Europe and South America.

Children and adolescents have not been exempt from this trend. Quite to the contrary, the increase in the USA has been substantial. Thus, between the 1960ies and 1990, overweight and obesity increased dramatically in 6 through to 17 year olds. The increments translate into relative increases of 40% using the 85^{th} BMI-centile (calculated in the 1960ies) as a cutoff and 100% upon use of the 95^{th} centile. In a cross sectional study of German children and adolescents treated as inpatients for extreme obesity between 1985 and 1995, a significant increase of the mean BMI of almost 2 kg/m² over this ten year period has been reported. Within this extreme group, the increments were most pronounced in the uppermost BMI ranges.

The mechanisms underlying this increase in the prevalence of obesity are unknown. Environmental factors have commonly been invoked as the underlying cause. Basically, both an increased caloric intake and a reduced level of physical activity have been discussed. In England the increase in obesity rates has been attributed to the latter mechanism. Thus, in this country, the average caloric intake even decreased somewhat within the last two decades, whereas indirect evidence stemming from the increases in hours spent watching television and from the average number of cars per household points to reduced levels of physical activity as the relevant causative factor.

Genetic factors have previously not been considered as a contributing cause. Quite to the contrary, the fact that the increased rates of obesity have been observed within the last two decades has been viewed as evidence that genetic factors cannot be held responsible. However, it has been proposed that an increase in the rate of assortative mating could very well constitute a genetic contribution to the observed phenomenon. This hypothesis is based on evidence suggesting that stigmatisation of obese individuals represents a rather recent social phenomenon, thus invariably having led to increased rates of assortative mating. As a consequence, the offspring have a higher loading with both additive and non-additive genetic factors underlying obesity. Indeed, an exceedingly high rate of (deduced) assortative mating amongst the parents of extremely obese children and adolescents has been observed.

Potentially life-threatening, chronic health problems associated with obesity fall into four main areas: 1) cardiovascular problems, including hypertension, chronic heart disease and stroke, 2) conditions associated with insulin resistance, namely Non-Insulin Dependent Diabetes Mellitus (NIDDM), 3) certain types of cancers, mainly the hormonally related and large-bowel cancers, and 4) gallbladder disease. Other problems associated with obesity include respiratory difficulties, chronic musculo-skeletal problems, skin problems and infertility (WHO, 1998).

The main currently available strategies for treating these disorders include dietary restriction, increments in physical activity, pharmacological and surgical approaches. In adults, long term weight loss is exceptional using conservative interventions. Present pharmacological interventions typically induce a weight loss of between five and fifteen kilograms; if the medication is discontinued, renewed weight gain ensues. Surgical treatments are comparatively successful and are reserved for patients with extreme obesity and/or with serious medical complications.

Recently, a 10 year old massively obese girl, in whom a leptin deficiency mutation had been detected, was treated successfully with recombinant leptin. This is the first individual who therapeutically profited from the detection of the mutation underlying her morbid obesity.

Several twin studies have been performed to estimate heritability of the BMI, some of which have encompassed over 1000 twin pairs. The results have been very consistent: The intrapair correlations among monozygotic twins were typically between 0.6 and 0.8, independent of age and gender. In one study, the correlations for monozygotic and dizygotic twins were basically the same, independent of whether the twins had been reared apart or together. Heritability of the BMI was estimated at 0.7; non-shared environmental factors explained the remaining 30% of the variance. Surprisingly, shared environmental factors did not explain a substantial proportion of the variance. Both hypercaloric and hypocaloric alimentation lead to similar degrees of weight gain or loss among both members of monozygotic twin pairs, indicating that genetic factors regulate the effect of environmentally induced variation of energy availability on body weight. Metabolic reactions and changes in body fat distribution upon overeating and undereating are also under genetic control (reviewed in Hebebrand et al., 1998).

A large adoption study has revealed that the BMI of adoptees is correlated with that of their biological parents and not with the BMI of the adoptive parents. Depending on the family study, the correlation between the BMI of sibs is between 0.2 and 0.4. Parent-offspring correlations are typically slightly lower. Segregation analyses have repeatedly suggested a major recessive gene effect. Based on these analyses, sample size calculations have been performed based on both concordant and discordant approaches. In contrast to the expectations, the concordant sib-pair approach was superior; a lower number of families were required to achieve the same power.

Family studies based on extremely obese young index patients, either mother or father or both, have a BMI>90^{th} decile in the vast majority of the families. Based on index patients with a BMI>95^{th} centile, approximately 20% of the respective families have a sib with a BMI>90^{th} centile.

In conclusion, it is apparent that environmental factors interact with specific genotypes rendering an individual more or less susceptible to the development of obesity. Furthermore, despite the fact that major genes have been detected, it is necessary to consider that the spectrum reaches from such major genes to genes with an only minor influence.

The discovery of the leptin gene at the end of 1994 (Zhang et al., 1994) has been followed by a virtual explosion of scientific efforts to uncover the regulatory systems underlying appetite and weight regulation. It is currently the fastest growing biomedical field. This upswing has also resulted in large scaled molecular genetic activities which, due to obvious clinical interest, are basically all related to obesity in humans, rodents and other mammals (Hebebrand et al., 1998).

In this respect, many genes in which mutations lead to the presently known monogenic forms of obesity have been cloned in rodents. Systemic consequences of these mutations are currently being analysed. These models have provided insights into the complex regulatory systems involved in body weight regulation, the best known of which includes leptin and its receptor.

In mice, but also in pigs, over 15 quantitative trait loci (QTL) have been identified that are most likely relevant in weight regulation (Chagnon et al., 2003).

In humans, four exceedingly rare autosomal recessive forms of obesity have been described as of 1997. Mutations in the genes encoding for leptin, leptin receptor, prohormone convertase 1 and pro-opiomelanocortin (POMC) have been shown to cause massive obesity of an early onset type, associated with hyperphagia. Distinct additional clinical (e.g. red hair, primary amenorrhea) and/or endocrinological abnormalities (e.g. markedly altered serum leptin levels, lack of ACTH secretion) pinpointed to the respective candidate genes. Both the monogenic animal models and the human monogenic forms have led to new insights into the complex system underlying body weight regulation.

Very recently, the first autosomal dominant form of obesity was described in humans. Two different mutations within the melanocortin-4 receptor gene (MC4R) were observed to lead to extreme obesity in probands heterozygous for these variants. In contrast to the aforementioned findings, these mutations do not implicate readily obvious phenotypic abnormalities other than extreme obesity (Vaisse et al., 1998; Yeo et al., 1998). Interestingly, both groups detected the mutations by systematic screens in relatively small study groups (n=63 and n=43).

Hinney et al. (1999) screened the MC4R in a total of 492 obese children and adolescents. All in all, four individuals with two different mutations leading to haplo-insufficiency were detected. One was identical to that previously observed by Yeo et al. (1998). The other mutation, which was detected in three individuals, induced a stop mutation in the extracellular domain of the receptor. Approximately one percent of extremely obese individuals harbour haplo-insufficiency mutations in the MC4R. In addition to the two forms of haplo-insufficiency, Hinney et al. (1999) also detected additional mutations leading to both conservative and non-conservative amino acid exchanges. Interestingly, these mutations were mainly observed in the obese study group. The functional implications of these mutations are currently unknown.

The identification of individuals with MC4R mutations is interesting in the light of possible pharmacological interventions. Thus, intranasal application of adrenocorticotropin₄₋₁₀ (ACTH₄₋₁₀), representing a core sequence of all melanocortins, resulted in reduced weight, body fat mass and plasma leptin concentrations in healthy controls. The question arises as to how mutation carriers would react to this treatment, which could theoretically counterbalance their reduced receptor density.

The involvement of specific genes in weight regulation is further substantiated by data obtained from transgenic mice. For example, MC4R deficient mice develop early onset obesity (Huszar et al., 1997).

Different groups are conducting genome scans related to obesity or dependent phenotypes (BMI, leptin levels, fat mass, etc.). This approach appears very promising, because it is both systematic and model free. In addition, it has already been shown to be exceptionally successful. Thus, positive linkage results have been obtained even by analysing comparatively small study groups. More important, some findings have already been replicated. Each of the following regions has been identified by at least two independent groups: chromosome 1p32, chromosome 2p21, chromosome 6p21, chromosome 10 and chromosome 20q13 (Chagnon et al., 2003).

### SUMMARY OF THE INVENTION

The present invention now discloses the identification of a human obesity susceptibility gene, which can be used for the diagnosis, prevention and treatment of obesity, and metabolic disorders, as well as for the screening of therapeutically active drugs.

The present invention more particularly discloses the identification of a human obesity susceptibility gene, which can be used for the diagnosis, prevention and treatment of obesity and associated disorders, as well as for the screening of therapeutically active drugs. The invention more specifically discloses certain alleles of the contactin associated protein-like 2 (CNTNAP2) gene related to susceptibility to obesity and representing novel targets for therapeutic intervention. The present invention relates to particular mutations in the CNTNAP2 gene and expression products, as well as to diagnostic tools and kits based on these mutations. The invention can be used in the diagnosis of predisposition to, detection, prevention and/or treatment of coronary heart disease and metabolic disorders, including but not limited to hypoalphalipoproteinemia, familial combined hyperlipidemia, insulin resistant syndrome X or multiple metabolic disorder, coronary artery disease, diabetes and associated complications and dyslipidemia.

The invention can be used in the diagnosis of predisposition to or protection from, detection, prevention and/or treatment of obesity or associated disorders, the method comprising detecting in a sample from the subject the presence of an alteration in the CNTNAP2 gene or polypeptide, the presence of said alteration being indicative of the presence or predisposition to obesity or associated disorders. The presence of said alteration can also be indicative for protecting against obesity.

A particular object of this invention resides in a method of detecting the presence of or predisposition to obesity or associated disorders in a subject, the method comprising detecting the presence of an alteration in the CNTNAP2 gene locus in a sample from the subject, the presence of said alteration being indicative of the presence of or the predisposition to obesity or associated disorders.

An additional particular object of this invention resides in a method of detecting the protection from obesity or associated disorders in a subject, the method comprising detecting the presence of an alteration in the CNTNAP2 gene locus in a sample from the subject, the presence of said alteration being indicative of the protection from obesity or associated disorders.

Another particular object of this invention resides in a method of assessing the response of a subject to a treatment of obesity or associated disorders, the method comprising detecting the presence of an alteration in the CNTNAP2 gene locus in a sample from the subject, the presence of said alteration being indicative of a particular response to said treatment.

A further particular object of this invention resides in a method of assessing the adverse effect in a subject to a treatment of obesity or associated disorders, the method comprising detecting the presence of an alteration in the CNTNAP2 gene locus in a sample from the subject, the presence of said alteration being indicative of an adverse effect to said treatment.

This invention also relates to a method for preventing obesity or an associated disorder in a subject, comprising detecting the presence of an alteration in the CNTNAP2 gene locus in a sample from the subject, the presence of said alteration being indicative of the predisposition to obesity or associated disorders; and, administering a prophylactic treatment against obesity or an associated disorder.

In a preferred embodiment, said alteration is one or several SNP(s) or a haplotype of SNPs associated with obesity or an associated disorder. More preferably, said haplotype associated with obesity or an associated disorder comprises or consists of several SNPs selected from the group consisting of SNP11, SNP14, SNP40, SNP45, SNP47, SNP48, SNP49, SNP50, SNP51, SNP53, SNP56, SNP60, SNP63, SNP65, SNP77, SNP94, SNP111, SNP112, SNP113, SNP116, SNP141, SNP142, SNP156, SNP161 and SNP163. Still more preferably, said haplotype is selected from the haplotypes disclosed in Table 4. More preferably, said SNP associated with obesity or an associated disorder can be selected from the group consisting of SNP45, SNP48, SNP49, SNP50, SNP51, SNP53, SNP56, SNP60, SNP63, SNP65, SNP77, SNP94, SNP111, SNP112, SNP141, SNP142, SNP156, SNP161 and SNP163. More preferably, said SNP associated with protection against obesity can be selected from the group consisting of SNP45, SNP48, SNP49, SNP50, SNP51, SNP53, SNP56, SNP60, SNP63, SNP65, SNP77, SNP94, SNP111, SNP112, SNP141, SNP142, SNP156, SNP161 and SNP163. In one particularly preferred embodiment, said SNP associated with protection against obesity can be SNP156, more particularly allele 2 of this SNP.

Preferably, the alteration in the CNTNAP2 gene locus is determined by performing a hydridization assay, a sequencing assay, a microsequencing assay, or an allele-specific amplification assay.

A particular aspect of this invention resides in compositions of matter comprising primers, probes, and/or oligonucleotides, which are designed to specifically detect at least one SNP or haplotype associated with obesity in the genomic region including the CNTNAP2 gene, or a combination thereof. More preferably, said haplotype associated with obesity or an associated disorder comprises or consists of several SNPs selected from the group consisting of SNP11, SNP14, SNP40, SNP45, SNP47, SNP48, SNP49, SNP50, SNP51, SNP53, SNP56, SNP60, SNP63, SNP65, SNP77, SNP94, SNP111, SNP112, SNP113, SNP116, SNP141, SNP142, SNP156, SNP161 and SNP163. Still more preferably, said haplotype is selected from the haplotypes disclosed in Table 4. More preferably, said SNP associated with obesity or an associated disorder can be selected from the group consisting of SNP45, SNP48, SNP49, SNP50, SNP51, SNP53, SNP56, SNP60, SNP63, SNP65, SNP77, SNP94, SNP111, SNP112, SNP141, SNP142, SNP156, SNP161 and SNP163. More preferably, said SNP associated with protection against obesity can be selected from the group consisting of SNP45, SNP48, SNP49, SNP50, SNP51, SNP53, SNP56, SNP60, SNP63, SNP65, SNP77, SNP94, SNP111, SNP112, SNP141, SNP142, SNP156, SNP161 and SNP163. In one particularly preferred embodiment, said SNP associated with protection against obesity can be SNP156, more particularly allele 2 of this SNP.

The invention also resides in methods of treating obesity and/or associated disorders in a subject through a modulation of CNTNAP2 expression or activity. Such treatments use, for instance, CNTNAP2 polypeptides, CNTNAP2 DNA sequences (including antisense sequences and RNAi directed at the CNTNAP2 gene locus), anti-CNTNAP2 antibodies or drugs that modulate CNTNAP2 expression or activity.

The invention also relates to methods of treating individuals who carry deleterious alleles of the CNTNAP2 gene, including pre-symptomatic treatment or combined therapy, such as through gene therapy, protein replacement therapy or through the administration of CNTNAP2 protein mimetics and/or inhibitors.

A further aspect of this invention resides in the screening of drugs for therapy of obesity or associated disorder, based on the modulation of or binding to an allele of CNTNAP2 gene associated with obesity or associated disorder or gene product thereof.

A further aspect of this invention includes antibodies specific of CNTNAP2 polypeptide fragments and derivatives of such antibodies, hybridomas secreting such antibodies, and diagnostic kits comprising those antibodies. More preferably, said antibodies are specific to a CNTNAP2 polypeptide or a fragment thereof comprising an alteration, said alteration modifying the activity of CNTNAP2.

The invention also concerns a CNTNAP2 gene or a fragment thereof comprising an alteration, said alteration modifying the activity of CNTNAP2. The invention further concerns a CNTNAP2 polypeptide or a fragment thereof comprising an alteration, said alteration modifying the activity of CNTNAP2.

### LEGEND TO THE FIGURES

FIG. 1: High density mapping using Genomic Hybrid Identity Profiling (GenomeHIP) A total of 2263 BAC clones with an average spacing of 1.2 Mega base pairs between clones representing the whole human genome were tested for linkage using GenomeHIP. Each point corresponds to a clone. Significant evidence for linkage was calculated for clone BACA2ZB02 (p-value 1.20E-07). The whole linkage region encompasses a region from 4 144 736 618 base pairs to 150 314 820 base pairs on human chromosome 7. The p-value 2×10⁻⁵ corresponding to the significance level for significant linkage was used as a significance level for whole genome screens as proposed by Lander and Kruglyak (1995).

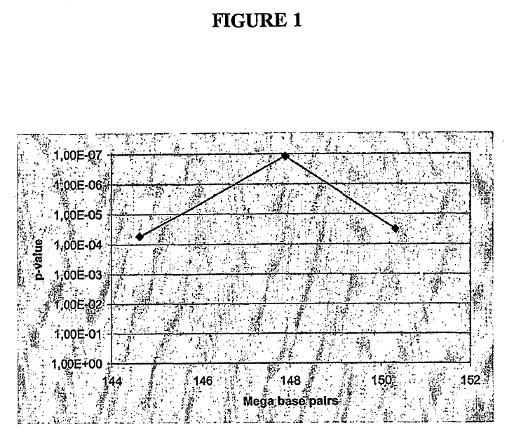

### DETAILED DESCRIPTION OF THE INVENTION

The present invention discloses the identification of CNTNAP2 as a human obesity susceptibility gene. Various nucleic acid samples from families with obesity were submitted to a particular GenomeHIP process. This process led to the identification of particular identical-by-descent fragments in said populations that are altered in obese subjects. By screening of the IBD fragments, we identified the contactin associated protein-like 2 gene on chromosome 7q35-q36 (CNTNAP2) as a candidate for obesity and related disorders. This gene is indeed present in the critical interval and expresses a functional phenotype consistent with a genetic regulation of obesity. SNPs of the CNTNAP2 gene were also identified, as being correlated to obesity in human subjects. SNP45, SNP47, SNP48, SNP49, SNP50, SNP51, SNP53, SNP56, SNP60, SNP63, SNP65, SNP77, SNP94, SNP111, SNP112, SNP141, SNP142, SNP156, SNP161 and SNP163, located in the CNTNAP2 gene locus were found to be associated with obesity or an associated disorder. Haplotypes disclosed in Table 4 comprising several SNPs selected from the group consisting of SNP11, SNP14, SNP40, SNP45, SNP48, SNP49, SNP50, SNP51, SNP53, SNP56, SNP60, SNP63, SNP65, SNP77, SNP94, SNP111, SNP112, SNP113, SNP116, SNP141, SNP142, SNP156, SNP161 and SNP163 have also been identified as associated with obesity. SNP156, more particularly allele 2, was also found to be associated with a protection against obesity.

The present invention thus proposes to use CNTNAP2 gene and corresponding expression products for the diagnosis, prevention and treatment of obesity and associated disorders, as well as for the screening of therapeutically active drugs.

### DEFINITIONS

Obesity and metabolic disorders: Obesity shall be construed as any condition of abnormal or excessive fat accumulation in adipose tissue, to the extent that health may be impaired. Associated disorders, diseases or pathologies include, more specifically, any metabolic disorders, including diabetes mellitus (more particularly type II diabetes) and associated complications such as diabetic neuropathy, hypo-alphalipoproteinemia, familial combined hyperlipidemia, hyperinsulinemia, insulin resistance, insulin resistant syndrome X or multiple metabolic disorder, cardiovascular complications such as coronary artery disease, and dyslipidemia. Preferred associated disorders are selected from the group consisting of type II diabetes, hyperinsulinemia, insulin resistance, and diabetic neuropathy.

The invention may be used in various subjects, particularly human, including adults, children and at the prenatal stage.

Within the context of this invention, the CNTNAP2 gene locus designates all CNTNAP2 sequences or products in a cell or organism, including CNTNAP2 coding sequences, CNTNAP2 non-coding sequences (e.g., introns), CNTNAP2 regulatory sequences controlling transcription and/or translation (e.g., promoter, enhancer, terminator, etc.), as well as all corresponding expression products, such as CNTNAP2 RNAs (e.g., mRNAs) and CNTNAP2 polypeptides (e.g., a pre-protein and a mature protein). The CNTNAP2 gene locus also comprise surrounding sequences of the CNTNAP2 gene which include SNPs that are in linkage disequilibrium with SNPs located in the CNTNAP2 gene. For example, the CNTNAP2 locus comprises surrounding sequences comprising SNP11, SNP14, SNP40, SNP45, SNP47, SNP48, SNP49, SNP50, SNP51, SNP53, SNP56, SNP60, SNP63, SNP65, SNP77, SNP94, SNP111, SNP112, SNP113, SNP116, SNP141, SNP142, SNP156, SNP161 and SNP163.

As used in the present application, the term “CNTNAP2 gene” designates the contactin associated protein-like 2 gene on chromosome 7q35-q36, as well as variants, analogs and fragments thereof, including alleles thereof (e.g., germline mutations) which are related to susceptibility to obesity and associated disorders. The CNTNAP2 gene may also be referred to as contactin-associated protein 2, cell recognition molecule (CASPR2), homolog of Drosophilia neurexin IV (NRXN4).

The term “gene” shall be construed to include any type of coding nucleic acid, including genomic DNA (gDNA), complementary DNA (cDNA), synthetic or semi-synthetic DNA, as well as any form of corresponding RNA. The term gene particularly includes recombinant nucleic acids encoding CNTNAP2, i.e., any non naturally occurring nucleic acid molecule created artificially, e.g., by assembling, cutting, ligating or amplifying sequences. A CNTNAP2 gene is typically double-stranded, although other forms may be contemplated, such as single-stranded. CNTNAP2 genes may be obtained from various sources and according to various techniques known in the art, such as by screening DNA libraries or by amplification from various natural sources. Recombinant nucleic acids may be prepared by conventional techniques, including chemical synthesis, genetic engineering, enzymatic techniques, or a combination thereof. Suitable CNTNAP2 gene sequences may be found on gene banks, such as Unigene Cluster for CNTNAP2 (Hs. 106552) and Unigene Representative Sequence NM_{—}014141. A particular example of a CNTNAP2 gene comprises SEQ ID No: 1.

The term “CNTNAP2 gene” includes any variant, fragment or analog of SEQ ID No 1 or of any coding sequence as identified above. Such variants include, for instance, naturally-occurring variants due to allelic variations between individuals (e.g., polymorphisms), mutated alleles related to obesity or an associated disorder, alternative splicing forms, etc. The term variant also includes CNTNAP2 gene sequences from other sources or organisms. Variants are preferably substantially homologous to SEQ ID No 1, i.e., exhibit a nucleotide sequence identity of at least about 65%, typically at least about 75%, preferably at least about 85%, more preferably at least about 95% with SEQ ID No 1. Variants and analogs of a CNTNAP2 gene also include nucleic acid sequences, which hybridize to a sequence as defined above (or a complementary strand thereof) under stringent hybridization conditions.

Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

A fragment of a CNTNAP2 gene designates any portion of at least about 8 consecutive nucleotides of a sequence as disclosed above, preferably at least about 15, more preferably at least about 20 nucleotides, further preferably of at least 30 nucleotides. Fragments include all possible nucleotide lengths between 8 and 100 nucleotides, preferably between 15 and 100, more preferably between 20 and 100.

A CNTNAP2 polypeptide designates any protein or polypeptide encoded by a CNTNAP2 gene as disclosed above. The term “polypeptide” refers to any molecule comprising a stretch of amino acids. This term includes molecules of various lengths, such as peptides and proteins. The polypeptide may be modified, such as by glycosylations and/or acetylations and/or chemical reaction or coupling, and may contain one or several non-natural or synthetic amino acids. A specific example of a CNTNAP2 polypeptide comprises all or part of SEQ ID No: 2 (NP_{—}054860).

The terms “response to a treatment” refer to treatment efficacy, including but not limited to ability to metabolise a therapeutic compound, to the ability to convert a prodrug to an active drug, and to the pharmacokinetics (absorption, distribution, elimination) and the pharmacodynamics (receptor-related) of a drug in an individual.

The terms “adverse effects to a treatment” refer to adverse effects of therapy resulting from extensions of the principal pharmacological action of the drug or to idiosyncratic adverse reactions resulting from an interaction of the drug with unique host factors. “Side effects to a treatment” include, but are not limited to, adverse reactions such as dermatologic, hematologic or hepatologic toxicities and further includes gastric and intestinal ulceration, disturbance in platelet function, renal injury, generalized urticaria, bronchoconstriction, hypotension, and shock.

### Diagnosis

The invention now provides diagnosis methods based on a monitoring of the CNTNAP2 gene locus in a subject. Within the context of the present invention, the term “diagnosis” includes the detection, monitoring, dosing, comparison, etc., at various stages, including early, pre-symptomatic stages, and late stages, in adults, children and pre-birth. Diagnosis typically includes the prognosis, the assessment of a predisposition or risk of development, the characterization of a subject to define most appropriate treatment (pharmacogenetics), etc.

The present invention provides diagnostic methods to determine whether an individual is at risk of developing obesity or an obesity-associated disorder or suffers from obesity or an obesity-associated disorder resulting from a mutation or a polymorphism in the CNTNAP2 gene locus. The present invention also provides methods to determine whether an individual is likely to respond positively to a therapeutic agent or whether an individual is at risk of developing an adverse side effect to a therapeutic agent.

A particular object of this invention resides in a method of detecting the presence of or predisposition to obesity or an obesity-associated disorder in a subject, the method comprising detecting in a sample from the subject the presence of an alteration in the CNTNAP2 gene locus in said sample. The presence of said alteration is indicative of the presence or predisposition to obesity or an obesity-associated disorder. Optionally, said method comprises a previous step of providing a sample from a subject. Preferably, the presence of an alteration in the CNTNAP2 gene locus in said sample is detected through the genotyping of a sample.

Another particular object of this invention resides in a method of detecting the protection from obesity or an obesity-associated disorder in a subject, the method comprising detecting the presence of an alteration in the CNTNAP2 gene locus in a sample from the subject, the presence of said alteration being indicative of the protection from obesity or an obesity-associated disorder.

In a preferred embodiment, said alteration is one or several SNP(s) or a haplotype of SNPs associated with obesity or an associated disorder. More preferably, said haplotype associated with obesity or an associated disorder comprises or consists of several SNPs selected from the group consisting of SNP11, SNP14, SNP40, SNP45, SNP47, SNP48, SNP49, SNP50, SNP51, SNP53, SNP56, SNP60, SNP63, SNP65, SNP77, SNP94, SNP111, SNP112, SNP113, SNP116, SNP141, SNP142, SNP156, SNP161 and SNP163. Still more preferably, said haplotype is selected from the haplotypes disclosed in Table 4. More preferably, said SNP associated with obesity or an associated disorder is selected from the group consisting of SNP45, SNP48, SNP49, SNP50, SNP51, SNP53, SNP56, SNP60, SNP63, SNP65, SNP77, SNP94, SNP111, SNP112, SNP141, SNP142, SNP156, SNP161 and SNP163. More preferably, said SNP associated with protection against obesity can be selected from the group consisting of SNP45, SNP48, SNP49, SNP50, SNP51, SNP53, SNP56, SNP60, SNP63, SNP65, SNP77, SNP94, SNP111, SNP112, SNP141, SNP142, SNP156, SNP161 and SNP163. In one particularly preferred embodiment, said SNP associated with protection against obesity can be SNP156, more particularly allele 2 of this SNP.

Another particular object of this invention resides in a method of assessing the response of a subject to a treatment of obesity or an associated disorder, the method comprising (i) providing a sample from the subject and (ii) detecting the presence of an alteration in the CNTNAP2 gene locus in said sample.

Another particular object of this invention resides in a method of assessing the response of a subject to a treatment of obesity or an associated disorder, the method comprising detecting in a sample from the subject the presence of an alteration in the CNTNAP2 gene locus in said sample. The presence of said alteration is indicative of a particular response to said treatment. Preferably, the presence of an alteration in the CNTNAP2 gene locus in said sample is detected through the genotyping of a sample.

A further particular object of this invention resides in a method of assessing the adverse effects of a subject to a treatment of obesity or an associated disorder, the method comprising detecting in a sample from the subject the presence of an alteration in the CNTNAP2 gene locus in said sample. The presence of said alteration is indicative of adverse effects to said treatment. Preferably, the presence of an alteration in the CNTNAP2 gene locus in said sample is detected through the genotyping of a sample.

In a preferred embodiment, said alteration is one or several SNP(s) or a haplotype of SNPs associated with obesity or an associated disorder. More preferably, said haplotype associated with obesity or an associated disorder comprises or consists of several SNPs selected from the group consisting of SNP11, SNP14, SNP40, SNP45, SNP47, SNP48, SNP49, SNP50, SNP51, SNP53, SNP56, SNP60, SNP63, SNP65, SNP77, SNP94, SNP111, SNP112, SNP113, SNP116, SNP141, SNP142, SNP156, SNP161 and SNP163. Still more preferably, said haplotype is selected from the haplotypes disclosed in Table 4. More preferably, said SNP associated with obesity or an associated disorder is selected from the group consisting of SNP45, SNP48, SNP49, SNP50, SNP51, SNP53, SNP56, SNP60, SNP63, SNP65, SNP77, SNP94, SNP111, SNP112, SNP141, SNP142, SNP156, SNP161 and SNP163. More preferably, said SNP associated with protection against obesity can be selected from the group consisting of SNP45, SNP48, SNP49, SNP50, SNP51, SNP53, SNP56, SNP60, SNP63, SNP65, SNP77, SNP94, SNP111, SNP112, SNP141, SNP142, SNP156, SNP161 and SNP163. In one particularly preferred embodiment, said SNP associated with protection against obesity can be SNP156, more particularly allele 2 of this SNP.

In an additional embodiment, the invention concerns a method for preventing obesity or an associated disorder in a subject, comprising detecting the presence of an alteration in the CNTNAP2 gene locus in a sample from the subject, the presence of said alteration being indicative of the predisposition to obesity or an associated disorder; and, administering a prophylactic treatment against obesity or an associated disorder. Said prophylactic treatment can be a drug administration and/or a diet.

Diagnostics, which analyse and predict response to a treatment or drug, or side effects to a treatment or drug, may be used to determine whether an individual should be treated with a particular treatment drug. For example, if the diagnostic indicates a likelihood that an individual will respond positively to treatment with a particular drug, the drug may be administered to the individual. Conversely, if the diagnostic indicates that an individual is likely to respond negatively to treatment with a particular drug, an alternative course of treatment may be prescribed. A negative response may be defined as either the absence of an efficacious response or the presence of toxic side effects.

Clinical drug trials represent another application for the CNTNAP2 SNPs. One or more CNTNAP2 SNPs indicative of response to a drug or to side effects to a drug may be identified using the methods described above. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

The alteration may be determined at the level of the CNTNAP2 gDNA, RNA or polypeptide. Optionally, the detection is performed by sequencing all or part of the CNTNAP2 gene or by selective hybridisation or amplification of all or part of the CNTNAP2 gene. More preferably a CNTNAP2 gene specific amplification is carried out before the alteration identification step.

An alteration in the CNTNAP2 gene locus may be any form of mutation(s), deletion(s), rearrangement(s) and/or insertions in the coding and/or non-coding region of the locus, alone or in various combination(s). Mutations more specifically include point mutations. Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The CNTNAP2 gene locus alteration may result in the creation of stop codons, frameshift mutations, amino acid substitutions, particular RNA splicing or processing, product instability, truncated polypeptide production, etc. The alteration may result in the production of a CNTNAP2 polypeptide with altered function, stability, targeting or structure. The alteration may also cause a reduction in protein expression or, alternatively, an increase in said production.

In a particular embodiment of the method according to the present invention, the alteration in the CNTNAP2 gene locus is selected from a point mutation, a deletion and an insertion in the CNTNAP2 gene or corresponding expression product, more preferably a point mutation and a deletion. The alteration may be determined at the level of the CNTNAP2 gDNA, RNA or polypeptide.

In this regard, the present invention now discloses SNPs in the CNTNAP2 gene and certain haplotypes, which include SNPs selected from the group consisting of SNP11, SNP14, SNP40, SNP45, SNP47, SNP48, SNP49, SNP50, SNP51, SNP53, SNP56, SNP60, SNP63, SNP65, SNP77, SNP94, SNP111, SNP112, SNP113, SNP116, SNP141, SNP142, SNP156, SNP161 and SNP163, that are associated with obesity or an associated disorder. The SNPs are reported in the following Table 1.

TABLE 1

| Position in chrom | | | | | Position in locus | |
|---|---|---|---|---|---|---|
| 7 based on NCBI | SNP | dbSNP | Allele | Allele | and type of amino | SEQ |
| Build 34 | identity | reference | 1 | 2 | acid change | ID No |
| 143467490 | 11 | rs1635079 | A | G | 5′ of CNTNAP2 | 3 |
| 143543795 | 14 | rs886469 | C | T | 5′ of CNTNAP2 | 4 |
| 145272638 | 40 | rs12703780 | A | G | intron | 5 |
| 145367704 | 45 | rs1405109 | C | T | intron | 6 |
| 145399533 | 47 | rs7806058 | A | G | intron | 7 |
| 145424265 | 48 | rs7788551 | C | T | intron | 8 |
| 145446406 | 49 | rs7809716 | A | G | intron | 9 |
| 145456858 | 50 | rs6464757 | C | T | intron | 10 |
| 145484824 | 51 | rs1639481 | A | G | intron | 11 |
| 145519990 | 53 | rs7784672 | A | G | intron | 12 |
| 145568121 | 56 | rs700273 | G | T | intron | 13 |
| 145654078 | 60 | rs12535870 | A | G | intron | 14 |
| 145709388 | 63 | rs1155384 | C | T | intron | 15 |
| 145734063 | 65 | rs7789802 | A | C | intron | 16 |
| 146043201 | 77 | rs13221567 | A | C | intron | 17 |
| 146312130 | 94 | Not | C | G | intron | 18 |
| | | available | | | | |
| 146603365 | 111 | rs851840 | C | G | intron | 19 |
| 146609747 | 112 | rs2888493 | A | G | intron | 20 |
| 146617641 | 113 | rs11764315 | A | C | intron | 21 |
| 146668312 | 116 | rs989242 | A | G | intron | 22 |
| 147225657 | 141 | rs2972125 | C | T | intron | 23 |
| 147254373 | 142 | rs963314 | A | G | intron | 24 |
| 147436213 | 156 | rs10278502 | C | T | intron | 25 |
| 147503860 | 161 | rs2270069 | C | T | intron | 26 |
| 147544244 | 163 | rs2717775 | C | G | 3′ of CNTNAP2 | 27 |

In any method according to the present invention, one or several SNP in the CNTNAP2 gene and certain haplotypes comprising SNP in the CNTNAP2 gene, more particularly SNP11, SNP14, SNP40, SNP45, SNP47, SNP48, SNP49, SNP50, SNP51, SNP53, SNP56, SNP60, SNP63, SNP65, SNP77, SNP94, SNP111, SNP112, SNP113, SNP116, SNP141, SNP142, SNP156, SNP161 and SNP163, can be used in combination with other SNP or haplotype associated with obesity or an associated disorder and located in other gene(s).

In another variant, the method comprises detecting the presence of an altered CNTNAP2 RNA expression. Altered RNA expression includes the presence of an altered RNA sequence, the presence of an altered RNA splicing or processing, the presence of an altered quantity of RNA, etc. These may be detected by various techniques known in the art, including by sequencing all or part of the CNTNAP2 RNA or by selective hybridisation or selective amplification of all or part of said RNA, for instance.

In a further variant, the method comprises detecting the presence of an altered CNTNAP2 polypeptide expression. Altered CNTNAP2 polypeptide expression includes the presence of an altered polypeptide sequence, the presence of an altered quantity of CNTNAP2 polypeptide, the presence of an altered tissue distribution, etc. These may be detected by various techniques known in the art, including by sequencing and/or binding to specific ligands (such as antibodies), for instance.

As indicated above, various techniques known in the art may be used to detect or quantify altered CNTNAP2 gene or RNA expression or sequence, including sequencing, hybridisation, amplification and/or binding to specific ligands (such as antibodies). Other suitable methods include allele-specific oligonucleotide (ASO), allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, heteroduplex analysis, RNase protection, chemical mismatch cleavage, ELISA, radio-immunoassays (RIA) and immuno-enzymatic assays (EMA).

Some of these approaches (e.g., SSCA and CGGE) are based on a change in electrophoretic mobility of the nucleic acids, as a result of the presence of an altered sequence. According to these techniques, the altered sequence is visualized by a shift in mobility on gels. The fragments may then be sequenced to confirm the alteration.

Some others are based on specific hybridisation between nucleic acids from the subject and a probe specific for wild type or altered CNTNAP2 gene or RNA. The probe may be in suspension or immobilized on a substrate. The probe is typically labeled to facilitate detection of hybrids.

Some of these approaches are particularly suited for assessing a polypeptide sequence or expression level, such as Northern blot, ELISA and RIA. These latter require the use of a ligand specific for the polypeptide, more preferably of a specific antibody.

In a particular, preferred, embodiment, the method comprises detecting the presence of an altered CNTNAP2 gene expression profile in a sample from the subject. As indicated above, this can be accomplished more preferably by sequencing, selective hybridisation and/or selective amplification of nucleic acids present in said sample.

### Sequencing

Sequencing can be carried out using techniques well known in the art, using automatic sequencers. The sequencing may be performed on the complete CNTNAP2 gene or, more preferably, on specific domains thereof, typically those known or suspected to carry deleterious mutations or other alterations.

### Amplification

Amplification is based on the formation of specific hybrids between complementary nucleic acid sequences that serve to initiate nucleic acid reproduction.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction.

Nucleic acid primers useful for amplifying sequences from the CNTNAP2 gene or locus are able to specifically hybridize with a portion of the CNTNAP2 gene locus that flank a target region of said locus, said target region being altered in certain subjects having obesity or an associated disorder. Examples of such target regions are provided in Table 1.

Primers that can be used to amplify CNTNAP2 target region comprising SNPs as identified in Table 1 may be designed based on the sequence of Seq Id No 1 or on the genomic sequence of CNTNAP2. In a particular embodiment, primers may be designed based on the sequence of SEQ ID Nos 3-27.

Another particular object of this invention resides in a nucleic acid primer useful for amplifying sequences from the CNTNAP2 gene or locus including surrounding regions. Such primers are preferably complementary to, and hybridize specifically to nucleic acid sequences in the CNTNAP2 gene locus. Particular primers are able to specifically hybridise with a portion of the CNTNAP2 gene locus that flank a target region of said locus, said target region being altered in certain subjects having obesity or an associated disorder.

The invention also relates to a nucleic acid primer, said primer being complementary to and hybridizing specifically to a portion of a CNTNAP2 coding sequence (e.g., gene or RNA) altered in certain subjects having obesity or an associated disorder. In this regard, particular primers of this invention are specific for altered sequences in a CNTNAP2 gene or RNA. By using such primers, the detection of an amplification product indicates the presence of an alteration in the CNTNAP2 gene locus. In contrast, the absence of amplification product indicates that the specific alteration is not present in the sample.

Typical primers of this invention are single-stranded nucleic acid molecules of about 5 to 60 nucleotides in length, more preferably of about 8 to about 25 nucleotides in length. The sequence can be derived directly from the sequence of the CNTNAP2 gene locus. Perfect complementarity is preferred, to ensure high specificity. However, certain mismatch may be tolerated.

The invention also concerns the use of a nucleic acid primer or a pair of nucleic acid primers as described above in a method of detecting the presence of or predisposition to obesity or an associated disorder in a subject or in a method of assessing the response of a subject to a treatment of obesity or an associated disorder.

### Selective Hybridization

Hybridization detection methods are based on the formation of specific hybrids between complementary nucleic acid sequences that serve to detect nucleic acid sequence alteration(s).

A particular detection technique involves the use of a nucleic acid probe specific for wild type or altered CNTNAP2 gene or RNA, followed by the detection of the presence of a hybrid. The probe may be in suspension or immobilized on a substrate or support (as in nucleic acid array or chips technologies). The probe is typically labeled to facilitate detection of hybrids.

In this regard, a particular embodiment of this invention comprises contacting the sample from the subject with a nucleic acid probe specific for an altered CNTNAP2 gene locus, and assessing the formation of an hybrid. In a particular, preferred embodiment, the method comprises contacting simultaneously the sample with a set of probes that are specific, respectively, for wild type CNTNAP2 gene locus and for various altered forms thereof. In this embodiment, it is possible to detect directly the presence of various forms of alterations in the CNTNAP2 gene locus in the sample. Also, various samples from various subjects may be treated in parallel.

Within the context of this invention, a probe refers to a polynucleotide sequence which is complementary to and capable of specific hybridisation with a (target portion of a) CNTNAP2 gene or RNA, and which is suitable for detecting polynucleotide polymorphisms associated with CNTNAP2 alleles which predispose to or are associated with obesity or an associated disorder. Probes are preferably perfectly complementary to the CNTNAP2 gene, RNA, or target portion thereof. Probes typically comprise single-stranded nucleic acids of between 8 to 1000 nucleotides in length, for instance of between 10 and 800, more preferably of between 15 and 700, typically of between 20 and 500. It should be understood that longer probes may be used as well. A preferred probe of this invention is a single stranded nucleic acid molecule of between 8 to 500 nucleotides in length, which can specifically hybridise to a region of a CNTNAP2 gene or RNA that carries an alteration.

A specific embodiment of this invention is a nucleic acid probe specific for an altered (e.g., a mutated) CNTNAP2 gene or RNA, i.e., a nucleic acid probe that specifically hybridises to said altered CNTNAP2 gene or RNA and essentially does not hybridise to a CNTNAP2 gene or RNA lacking said alteration. Specificity indicates that hybridisation to the target sequence generates a specific signal which can be distinguished from the signal generated through non-specific hybridisation. Perfectly complementary sequences are preferred to design probes according to this invention. It should be understood, however, that certain a certain degree of mismatch may be tolerated, as long as the specific signal may be distinguished from non-specific hybridisation.

Particular examples of such probes are nucleic acid sequences complementary to a target portion of the genomic region including the CNTNAP2 gene or RNA carrying a point mutation as listed in Table 1 above. More particularly, the probes can comprise a sequence selected from the group consisting of SEQ ID Nos 3-27 or a fragment thereof comprising the SNP or a complementary sequence thereof.

The sequence of the probes can be derived from the sequences of the CNTNAP2 gene and RNA as provided in the present application. Nucleotide substitutions may be performed, as well as chemical modifications of the probe. Such chemical modifications may be accomplished to increase the stability of hybrids (e.g., intercalating groups) or to label the probe. Typical examples of labels include, without limitation, radioactivity, fluorescence, luminescence, enzymatic labeling, etc.

The invention also concerns the use of a nucleic acid probe as described above in a method of detecting the presence of or predisposition to obesity or an associated disorder in a subject or in a method of assessing the response of a subject to a treatment of obesity or an associated disorder.

### Specific Ligand Binding

As indicated above, alteration in the CNTNAP2 gene locus may also be detected by screening for alteration(s) in CNTNAP2 polypeptide sequence or expression levels. In this regard, a specific embodiment of this invention comprises contacting the sample with a ligand specific for a CNTNAP2 polypeptide and determining the formation of a complex.

Different types of ligands may be used, such as specific antibodies. In a specific embodiment, the sample is contacted with an antibody specific for a CNTNAP2 polypeptide and the formation of an immune complex is determined. Various methods for detecting an immune complex can be used, such as ELISA, radioimmunoassays (RIA) and immuno-enzymatic assays (IEMA).

Within the context of this invention, an antibody designates a polyclonal antibody, a monoclonal antibody, as well as fragments or derivatives thereof having substantially the same antigen specificity. Fragments include Fab, Fab′2, CDR regions, etc. Derivatives include single-chain antibodies, humanized antibodies, poly-functional antibodies, etc.

An antibody specific for a CNTNAP2 polypeptide designates an antibody that selectively binds a CNTNAP2 polypeptide, namely, an antibody raised against a CNTNAP2 polypeptide or an epitope-containing fragment thereof. Although non-specific binding towards other antigens may occur, binding to the target CNTNAP2 polypeptide occurs with a higher affinity and can be reliably discriminated from non-specific binding.

In a specific embodiment, the method comprises contacting a sample from the subject with (a support coated with) an antibody specific for an altered form of a CNTNAP2 polypeptide, and determining the presence of an immune complex. In a particular embodiment, the sample may be contacted simultaneously, or in parallel, or sequentially, with various (supports coated with) antibodies specific for different forms of a CNTNAP2 polypeptide, such as a wild type and various altered forms thereof.

The invention also concerns the use of a ligand, preferably an antibody, a fragment or a derivative thereof as described above, in a method of detecting the presence of or predisposition to obesity or an associated disorder in a subject or in a method of assessing the response of a subject to a treatment of obesity or an associated disorder.

The invention also relates to a diagnostic kit comprising products and reagents for detecting in a sample from a subject the presence of an alteration in the CNTNAP2 gene or polypeptide, in the CNTNAP2 gene or polypeptide expression, and/or in CNTNAP2 activity. Said diagnostic kit according to the present invention comprises any primer, any pair of primers, any nucleic acid probe and/or any ligand, preferably antibody, described in the present invention. Said diagnostic kit according to the present invention can further comprise reagents and/or protocols for performing a hybridization, amplification or antigen-antibody immune reaction.

The diagnosis methods can be performed in vitro, ex vivo or in vivo, preferably in vitro or ex vivo. They use a sample from the subject, to assess the status of the CNTNAP2 gene locus. The sample may be any biological sample derived from a subject, which contains nucleic acids or polypeptides. Examples of such samples include fluids, tissues, cell samples, organs, biopsies, etc. Most preferred samples are blood, plasma, saliva, urine, seminal fluid, etc. Pre-natal diagnosis may also be performed by testing fetal cells or placental cells, for instance. The sample may be collected according to conventional techniques and used directly for diagnosis or stored. The sample may be treated prior to performing the method, in order to render or improve availability of nucleic acids or polypeptides for testing. Treatments include, for instant, lysis (e.g., mechanical, physical, chemical, etc.), centrifugation, etc. Also, the nucleic acids and/or polypeptides may be pre-purified or enriched by conventional techniques, and/or reduced in complexity. Nucleic acids and polypeptides may also be treated with enzymes or other chemical or physical treatments to produce fragments thereof. Considering the high sensitivity of the claimed methods, very few amounts of sample are sufficient to perform the assay.

As indicated, the sample is preferably contacted with reagents such as probes, primers or ligands in order to assess the presence of an altered CNTNAP2 gene locus. Contacting may be performed in any suitable device, such as a plate, tube, well, glass, etc. In specific embodiments, the contacting is performed on a substrate coated with the reagent, such as a nucleic acid array or a specific ligand array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids or polypeptides of the sample.

The finding of an altered CNTNAP2 polypeptide, RNA or DNA in the sample is indicative of the presence of an altered CNTNAP2 gene locus in the subject, which can be correlated to the presence, predisposition or stage of progression of obesity or an associated disorder. For example, an individual having a germ line CNTNAP2 mutation has an increased risk of developing obesity or an associated disorder. The determination of the presence of an altered CNTNAP2 gene locus in a subject also allows the design of appropriate therapeutic intervention, which is more effective and customized. Also, this determination at the pre-symptomatic level allows a preventive regimen to be applied.

### Linkage Disequilibrium

Once a first SNP has been identified in a genomic region of interest, more particularly in CNTNAP2 gene locus, the practitioner of ordinary skill in the art can easily identify additional SNPs in linkage disequilibrium with this first SNP. Indeed, any SNP in linkage disequilibrium with a first SNP associated with obesity or an associated disorder will be associated with this trait. Therefore, once the association has been demonstrated between a given SNP and obesity or an associated disorder, the discovery of additional SNPs associated with this trait can be of great interest in order to increase the density of SNPs in this particular region.

Identification of additional SNPs in linkage disequilibrium with a given SNP involves: (a) amplifying a fragment from the genomic region comprising or surrounding a first SNP from a plurality of individuals; (b) identifying of second SNPs in the genomic region harboring or surrounding said first SNP; (c) conducting a linkage disequilibrium analysis between said first SNP and second SNPs; and (d) selecting said second SNPs as being in linkage disequilibrium with said first marker. Subcombinations comprising steps (b) and (c) are also contemplated.

Methods to identify SNPs and to conduct linkage disequilibrium analysis can be carried out by the skilled person without undue experimentation by using well-known methods.

These SNPs in linkage disequilibrium can also be used in the methods according to the present invention, and more particularly in the diagnostic methods according to the present invention.

For example, a linkage locus of Crohn's disease has been mapped to a large region spanning 18cM on chromosome 5q31 (Rioux et al., 2000 and 2001). Using dense maps of microsatellite markers and SNPs across the entire region, strong evidence of linkage disequilibrium (LD) was found. Having found evidence of LD, the authors developed an ultra-high-density SNP map and studied a denser collection of markers selected from this map. Multilocus analyses defined a single common risk haplotype characterised by multiple SNPs that were each independently associated using TDT. These SNPs were unique to the risk haplotype and essentially identical in their information content by virtue of being in nearly complete LD with one another. The equivalent properties of these SNPs make it impossible to identify the causal mutation within this region on the basis of genetic evidence alone.

### Causal Mutation

Mutations in the CNTNAP2 gene which are responsible for obesity or an associated disorder may be identified by comparing the sequences of the CNTNAP2 gene from patients presenting obesity or an associated disorder and control individuals. Based on the identified association of SNPs of CNTNAP2 and obesity or an associated disorder, the identified locus can be scanned for mutations. In a preferred embodiment, functional regions such as exons and splice sites, promoters and other regulatory regions of the CNTNAP2 gene are scanned for mutations. Preferably, patients presenting obesity or an associated disorder carry the mutation shown to be associated with obesity or an associated disorder and controls individuals do not carry the mutation or allele associated with obesity or an associated disorder. It might also be possible that patients presenting obesity or an associated disorder carry the mutation shown to be associated with obesity or an associated disorder with a higher frequency than controls individuals.

The method used to detect such mutations generally comprises the following steps: amplification of a region of the CNTNAP2 gene comprising a SNP or a group of SNPs associated with obesity or an associated disorder from DNA samples of the CNTNAP2 gene from patients presenting obesity or an associated disorder and control individuals; sequencing of the amplified region; comparison of DNA sequences of the CNTNAP2 gene from patients presenting obesity or an associated disorder and control individuals; determination of mutations specific to patients presenting obesity or an associated disorder.

Therefore, identification of a causal mutation in the CNTNAP2 gene can be carried out by the skilled person without undue experimentation by using well-known methods.

For example, the causal mutations have been identified in the following examples by using routine methods.

Hugot et al. (2001) applied a positional cloning strategy to identify gene variants with susceptibly to Crohn's disease in a region of chromosome 16 previously found to be linked to susceptibility to Crohn's disease. To refine the location of the potential susceptibility locus 26 microsatellite markers were genotyped and tested for association to Crohn's disease using the transmission disequilibrium test. A borderline significant association was found between one allele of the microsatellite marker D16S136. Eleven additional SNPs were selected from surrounding regions and several SNPs showed significant association. SNP5-8 from this region were found to be present in a single exon of the NOD2/CARD15 gene and shown to be non-synonymous variants. This prompted the authors to sequence the complete coding sequence of this gene in 50 CD patients. Two additional non-synonymous mutations (SNP12 and SNP13) were found. SNP13 was most significant associated (p=6×10−6) using the pedigree transmission disequilibrium test. In another independent study, the same variant was found also by sequencing the coding region of this gene from 12 affected individuals compared to 4 controls (Ogura et al., 2001). The rare allele of SNP13 corresponded to a 1-bp insertion predicted to truncate the NOD2/CARD15 protein. This allele was also present in normal healthy individuals, albeit with significantly lower frequency as compared to the controls.

Similarly, Lesage et al. (2002) performed a mutational analyses of CARD15 in 453 patients with CD, including 166 sporadic and 287 familial cases, 159 patients with ulcerative colitis (UC), and 103 healthy control subjects by systematic sequencing of the coding region. Of 67 sequence variations identified, 9 had an allele frequency >5% in patients with CD. Six of them were considered to be polymorphisms, and three (SNP12-R702W, SNP8-G908R, and SNP13-1007fs) were confirmed to be independently associated with susceptibility to CD. Also considered as potential disease-causing mutations (DCMs) were 27 rare additional mutations. The three main variants (R702W, G908R, and 1007fs) represented 32%, 18%, and 31%, respectively, of the total CD mutations, whereas the total of the 27 rare mutations represented 19% of DCMs. Altogether, 93% of the mutations were located in the distal third of the gene. No mutations were found to be associated with UC. In contrast, 50% of patients with CD carried at least one DCM, including 17% who had a double mutation.

### Drug Screening

The present invention also provides novel targets and methods for the screening of drug candidates or leads. The methods include binding assays and/or functional assays, and may be performed in vitro, in cell systems, in animals, etc.

A particular object of this invention resides in a method of selecting compounds active on obesity or an associated disorder, said method comprising contacting in vitro a test compound with a CNTNAP2 gene or polypeptide according to the present invention and determining the ability of said test compound to bind said CNTNAP2 gene or polypeptide. Binding to said gene or polypeptide provides an indication as to the ability of the compound to modulate the activity of said target, and thus to affect a pathway leading to obesity or an associated disorder in a subject. In a preferred embodiment, the method comprises contacting in vitro a test compound with a CNTNAP2 polypeptide or a fragment thereof according to the present invention and determining the ability of said test compound to bind said CNTNAP2 polypeptide or fragment. The fragment preferably comprises a binding site of the CNTNAP2 polypeptide. Preferably, said CNTNAP2 gene or polypeptide or a fragment thereof is an altered or mutated CNTNAP2 gene or polypeptide or a fragment thereof comprising the alteration or mutation.

A particular object of this invention resides in a method of selecting compounds active on obesity or an associated disorder, said method comprising contacting in vitro a test compound with a CNTNAP2 polypeptide according to the present invention or binding site-containing fragment thereof and determining the ability of said test compound to bind said CNTNAP2 polypeptide or fragment thereof. Preferably, said CNTNAP2 polypeptide or a fragment thereof is an altered or mutated CNTNAP2 polypeptide or a fragment thereof comprising the alteration or mutation.

In a further particular embodiment, the method comprises contacting a recombinant host cell expressing a CNTNAP2 polypeptide according to the present invention with a test compound, and determining the ability of said test compound to bind said CNTNAP2 and to modulate the activity of CNTNAP2 polypeptide. Preferably, said CNTNAP2 polypeptide or a fragment thereof is an altered or mutated CNTNAP2 polypeptide or a fragment thereof comprising the alteration or mutation.

The determination of binding may be performed by various techniques, such as by labeling of the test compound, by competition with a labeled reference ligand, etc.

A further object of this invention resides in a method of selecting compounds active on obesity or an associated disorder, said method comprising contacting in vitro a test compound with a CNTNAP2 polypeptide according to the present invention and determining the ability of said test compound to modulate the activity of said CNTNAP2 polypeptide. Preferably, said CNTNAP2 polypeptide or a fragment thereof is an altered or mutated CNTNAP2 polypeptide or a fragment thereof comprising the alteration or mutation.

A further object of this invention resides in a method of selecting compounds active on obesity or an associated disorder, said method comprising contacting in vitro a test compound with a CNTNAP2 gene according to the present invention and determining the ability of said test compound to modulate the expression of said CNTNAP2 gene. Preferably, said CNTNAP2 gene or a fragment thereof is an altered or mutated CNTNAP2 gene or a fragment thereof comprising the alteration or mutation.

In an other embodiment, this invention relates to a method of screening, selecting or identifying active compounds, particularly compounds active on obesity or an associated disorder, the method comprising contacting a test compound with a recombinant host cell comprising a reporter construct, said reporter construct comprising a reporter gene under the control of a CNTNAP2 gene promoter, and selecting the test compounds that modulate (e.g. stimulate or reduce) expression of the reporter gene. Preferably, said CNTNAP2 gene promoter or a fragment thereof is an altered or mutated CNTNAP2 gene promoter or a fragment thereof comprising the alteration or mutation.

In a particular embodiment of the methods of screening, the modulation is an inhibition. In another particular embodiment of the methods of screening, the modulation is an activation.

The above screening assays may be performed in any suitable device, such as plates, tubes, dishes, flasks, etc. Typically, the assay is performed in multi-wells plates. Several test compounds can be assayed in parallel. Furthermore, the test compound may be of various origin, nature and composition. It may be any organic or inorganic substance, such as a lipid, peptide, polypeptide, nucleic acid, small molecule, etc., in isolated or in mixture with other substances. The compounds may be all or part of a combinatorial library of products, for instance.

### Pharmaceutical Composition, Therapy

A further object of this invention is a pharmaceutical composition comprising (i) a CNTNAP2 polypeptide or a fragment thereof, a nucleic acid encoding a CNTNAP2 polypeptide or a fragment thereof, a vector or a recombinant host cell as described above and (ii) a pharmaceutically acceptable carrier or vehicle.

The invention also relates to a method of treating or preventing obesity or an associated disorder in a subject, the method comprising administering to said subject a functional (e.g., wild-type) CNTNAP2 polypeptide or a nucleic acid encoding the same.

An other embodiment of this invention resides in a method of treating or preventing obesity or an associated disorder in a subject, the method comprising administering to said subject a compound that modulates, preferably that activates or mimics, expression or activity of a CNTNAP2 gene or protein according to the present invention. Said compound can be an agonist or an antagonist of CNTNAP2, an antisense or a RNAi of CNTNAP2, an antibody or a fragment or a derivative thereof specific to a CNTNAP2 polypeptide according to the present invention. In a particular embodiment of the method, the modulation is an inhibition. In another particular embodiment of the method, the modulation is an activation.

The invention also relates, generally, to the use of a functional CNTNAP2 polypeptide, a nucleic acid encoding the same, or a compound that modulates expression or activity of a CNTNAP2 gene or protein according to the present invention, in the manufacture of a pharmaceutical composition for treating or preventing obesity or an associated disorder in a subject. Said compound can be an agonist or an antagonist of CNTNAP2, an antisense or a RNAi of CNTNAP2, an antibody or a fragment or a derivative thereof specific to a CNTNAP2 polypeptide according to the present invention. In a particular embodiment of the method, the modulation is an inhibition. In another particular embodiment of the method, the modulation is an activation.

The present invention demonstrates the correlation between obesity or an associated disorder and the CNTNAP2 gene locus. The invention thus provides a novel target of therapeutic intervention. Various approaches can be contemplated to restore or modulate the CNTNAP2 activity or function in a subject, particularly those carrying an altered CNTNAP2 gene locus. Supplying wild-type function to such subjects is expected to suppress phenotypic expression of obesity and associated disorders in a pathological cell or organism. The supply of such function can be accomplished through gene or protein therapy, or by administering compounds that modulate or mimic CNTNAP2 polypeptide activity (e.g., agonists as identified in the above screening assays).

The wild-type CNTNAP2 gene or a functional part thereof may be introduced into the cells of the subject in need thereof using a vector as described above. The vector may be a viral vector or a plasmid. The gene may also be introduced as naked DNA. The gene may be provided so as to integrate into the genome of the recipient host' cells, or to remain extra-chromosomal. Integration may occur randomly or at precisely defined sites, such as through homologous recombination. In particular, a functional copy of the CNTNAP2 gene may be inserted in replacement of an altered version in a cell, through homologous recombination. Further techniques include gene gun, liposome-mediated transfection, cationic lipid-mediated transfection, etc. Gene therapy may be accomplished by direct gene injection, or by administering ex vivo prepared genetically modified cells expressing a functional CNTNAP2 polypeptide.

Other molecules with CNTNAP2 activity (e.g., peptides, drugs, CNTNAP2 agonists, or organic compounds) may also be used to restore functional CNTNAP2 activity in a subject or to suppress the deleterious phenotype in a cell.

Restoration of functional CNTNAP2 gene function in a cell may be used to prevent the development of obesity or an associated disorder or to reduce progression of said diseases. Such a treatment may suppress the obesity-associated phenotype of a cell, particularly those cells carrying a deleterious allele.

Further aspects and advantages of the present invention will be disclosed in the following experimental section, which should be regarded as illustrative and not limiting the scope of the present application.

### Gene, Vectors, Recombinant Cells and Polypeptides

A further aspect of this invention resides in novel products for use in diagnosis, therapy or screening. These products comprise nucleic acid molecules encoding a CNTNAP2 polypeptide or a fragment thereof, vectors comprising the same, recombinant host cells and expressed polypeptides.

More particularly, the invention concerns an altered or mutated CNTNAP2 gene or a fragment thereof comprising said alteration or mutation. The invention also concerns nucleic acid molecules encoding an altered or mutated CNTNAP2 polypeptide or a fragment thereof comprising said alteration or mutation. Said alteration or mutation modifies the CNTNAP2 activity. The modified activity can be increased or decreased. The invention further concerns a vector comprising an altered or mutated CNTNAP2 gene or a fragment thereof comprising said alteration or mutation or a nucleic acid molecule encoding an altered or mutated CNTNAP2 polypeptide or a fragment thereof comprising said alteration or mutation, recombinant host cells and expressed polypeptides.

A further object of this invention is a vector comprising a nucleic acid encoding a CNTNAP2 polypeptide according to the present invention. The vector may be a cloning vector or, more preferably, an expression vector, i.e., a vector comprising regulatory sequences causing expression of a CNTNAP2 polypeptide from said vector in a competent host cell.

These vectors can be used to express a CNTNAP2 polypeptide in vitro, ex vivo or in vivo, to create transgenic or “Knock Out” non-human animals, to amplify the nucleic acids, to express antisense RNAs, etc.

The vectors of this invention typically comprise a CNTNAP2 coding sequence according to the present invention operably linked to regulatory sequences, e.g., a promoter, a polyA, etc. The term “operably linked” indicates that the coding and regulatory sequences are functionally associated so that the regulatory sequences cause expression (e.g., transcription) of the coding sequences. The vectors may further comprise one or several origins of replication and/or selectable markers. The promoter region may be homologous or heterologous with respect to the coding sequence, and may provide for ubiquitous, constitutive, regulated and/or tissue specific expression, in any appropriate host cell, including for in vivo use. Examples of promoters include bacterial promoters (T7, pTAC, Trp promoter, etc.), viral promoters (LTR, TK, CMV-IE, etc.), mammalian gene promoters (albumin, PGK, etc), and the like.

The vector may be a plasmid, a virus, a cosmid, a phage, a BAC, a YAC, etc. Plasmid vectors may be prepared from commercially available vectors such as pBluescript, pUC, pBR, etc. Viral vectors may be produced from baculoviruses, retroviruses, adenoviruses, AAVs, etc., according to recombinant DNA techniques known in the art.

In this regard, a particular object of this invention resides in a recombinant virus encoding a CNTNAP2 polypeptide as defined above. The recombinant virus is preferably replication-defective, even more preferably selected from E1- and/or E4-defective adenoviruses, Gag-, pol- and/or env-defective retroviruses and Rep- and/or Cap-defective AAVs. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. No. 5,882,877, U.S. Pat. No. 6,013,516, U.S. Pat. No. 4,861,719, U.S. Pat. No. 5,278,056 and WO94/19478.

A further object of the present invention resides in a recombinant host cell comprising a recombinant CNTNAP2 gene or a vector as defined above. Suitable host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from fibroblasts, embryonic cells, epithelial cells, nervous cells, adipocytes, etc.).

The present invention also relates to a method for producing a recombinant host cell expressing a CNTNAP2 polypeptide according to the present invention, said method comprising (i) introducing in vitro or ex vivo into a competent host cell a recombinant nucleic acid or a vector as described above, (ii) culturing in vitro or ex vivo the recombinant host cells obtained and (iii), optionally, selecting the cells which express the CNTNAP2 polypeptide.

Such recombinant host cells can be used for the production of CNTNAP2 polypeptides, as well as for screening of active molecules, as described below. Such cells may also be used as a model system to study obesity and associated disorders. These cells can be maintained in suitable culture media, such as DMEM, RPMI, HAM, etc., in any appropriate culture device (plate, flask, dish, tube, pouch, etc.).

### EXAMPLES

### 1. GenomeHIP Platform to Identify the Chromosome 7 Susceptibility Gene

The GenomeHIP platform was applied to allow rapid identification of an obesity susceptibility gene.

Briefly, the technology consists of forming pairs from the DNA of related individuals. Each DNA is marked with a specific label allowing its identification. Hybrids are then formed between the two DNAs. A particular process (WO00/53802) is then applied that selects all fragments identical-by-descent (IBD) from the two DNAs in a multi step procedure. The remaining IBD enriched DNA is then scored against a BAC clone derived DNA microarray that allows the positioning of the IBD fraction on a chromosome.

The application of this process over many different families results in a matrix of IBD fractions for each pair from each family. Statistical analyses then calculate the minimal IBD regions that are shared between all families tested. Significant results (p-values) are evidence for linkage of the positive region with the trait of interest (here obesity). The linked interval can be delimited by the two most distant clones showing significant p-values.

In the present study, 164 families of German origin (178 independent sib-pairs) concordant for massive obesity (as defined by a body mass index >90th % ile) were submitted to the GenomeHIP process. The resulting IBD enriched DNA fractions were then labelled with Cy5 fluorescent dyes and hybridised against a DNA array consisting of 2263 BAC clones covering the whole human genome with an average spacing of 1.2 Mega base pairs. Non-selected DNA labelled with Cy3 was used to normalize the signal values and compute ratios for each clone. Clustering of the ratio results was then performed to determine the IBD status for each clone and pair.

By applying this procedure, several BAC clones (BACA25ZB12, BACA2ZB02 and BACA20ZB03) spanning approximately 5.7 Mega bases in the region on chromosome 7 (bases 144 608 830 to 150 454 591) were identified, that showed significant evidence for linkage to obesity (p=1.20E-07).

Table 2: Linkage results for chromosome 7 in the CNTNAP2 locus: Indicated is the region correspondent to 3 BAC clones with evidence for linkage. The start and stop positions of the clones correspond to their genomic location based on NCBI Build34 sequence respective to the start of the chromosome (p-ter).

TABLE 2

| | | | | Proportion of | |
|---|---|---|---|---|---|
| Human | | | | informative | |
| chromosome | Clones | Start | End | pairs | p-value |
| 7 | BACA25ZB12 | 144 608 830 | 144 736 618 | 0.92 | 5.40E−05 |
| 7 | BACA2ZB02 | 147 907 717 | 148 076 897 | 0.88 | 1.20E−07 |
| 7 | BACA20ZB03 | 150 314 820 | 150 454 591 | 0.88 | 3.20E−05 |

### 2. Identification of an Obesity Susceptibility Gene on Chromosome 7

By screening the aforementioned 5.7 Megabases in the linked chromosomal region, we identified the contactin associated protein-like 2 (CNTNAP2) gene as a candidate for obesity and related phenotypes. This gene is indeed present in the critical interval, with evidence for linkage delimited by the clones outlined above.

CNTNAP2 gene encodes a predicted 1331-amino acid polypeptide for NP_{—}054860 (mRNA NM_{—}014141, 8107 bp) and spreads over 2304.258 kb of genomic sequence. The protein encoded by this gene is a member of the neurexin family which functions in the vertebrate nervous system as cell adhesion molecules and receptors. This protein, like other neurexin proteins, contains epidermal growth factor repeats and laminin G domains. In addition, it includes an F5/8 type C domain, discoidin/neuropilin- and fibrinogen-like domains, thrombospondin N-terminal-like domains and a putative PDZ binding site. This protein is localized at the juxtaparanodes of myelinated axons and associated with potassium channels.

CNTNAP2 precisely colocalized with Shaker-like potassium channels in the juxtaparanodal region (Poliak et al., 1999). CNTNAP2 specifically associated with Kv1.1 (KCNA1), Kv1.2 (KCNA2), and their Kv-beta-2 subunit (KCNA2). This association involved the C-terminal region of CNTNAP2. This lead the authors to suggest that CNTNAP2 may stabilize the localization of potassium channels in the juxtaparanodal region, and that CNTNAP2 family members may play a role in the local differentiation of the axon into distinct functional subdomains.

Significant linkage and association with KCNAB2 and KCNA1 was found in the same families as used in the present study (see patent application U.S. 60/578,829) indicating that a set of proteins interacting with each other are jointly contributing to the development of obesity.

Voltage-gated potassium (Kv) channels represent the most complex class of voltage-gated ion channels from both functional and structural standpoints. Their diverse functions include regulating neurotransmitter release, heart rate, insulin secretion, neuronal excitability, epithelial electrolyte transport, smooth muscle contraction, and cell volume.

Recent investigations suggest that Kv channels are active participants in the regulation of beta-cell electrical activity and insulin secretion (MacDonald and Wheeler, 2003). Beta-cell Kv channels are targets of the G-protein coupled GLP-1 receptor and signals from glucose metabolism, pathways which could be physiologically relevant to the control of insulin secretion (MacDonald and Wheeler, 2003).

Examination of Kv1.3-deficient mice (Kv1.3(−/−)) revealed a previously unrecognized role for Kv1.3 in body weight regulation. Kv1.3(−/−) mice weighed significantly less than control littermates (Xu et al., 2003). Moreover, knockout mice were protected from diet-induced obesity and gained significantly less weight than littermate controls when placed on a high-fat diet.

McDaniel et al. (2001) reported an anorexic effect of K+ channel blockade by extracellular application of 4-aminopyridine (4-AP), a Kv-channel blocker, in mesenteric arterial smooth muscle (MASMC) and intestinal epithelial cells functionally expressing multiple Kv channel alpha- and beta-subunits including Kvbeta2.1 encoded by KCNAB2 in rats.

It has been demonstrated that the anorexic drugs, fenfluramine and dexenfluramine, in addition to inhibiting serotonin transporters (Baumann et al., 2000), decrease Kv channel activity in vascular smooth muscle cells (Hu et al., 1998, Michelakis et al., 1999; Wang et al., 1997). These observations suggest that the activity of Kv channels in MASMC may play an important role in the regulation of energy intake by controlling nutrient transportation.

Taken together, the linkage results provided in the present application, identifying the human CNTNAP2 gene in the critical interval of genetic alterations linked to obesity on chromosome 7, with its involvement in the interaction with voltage-gated potassium (Kv) channels, we conclude that alterations (e.g., mutations and/or polymorphisms) in the CNTNAP2 gene or its regulatory sequences may contribute to the development of human obesity and represent a novel target for diagnosis or therapeutic intervention.

### 3. Association Study

The same families that have been used for the linkage study were also used to test for association between a specific phenotype (here obesity) in question and the genetic marker allele or haplotypes containing a specific marker allele using the transmission disequilibrium test (TDT). The TDT is a powerful association test as it is insensitive to population stratification problems in the tested sample. Briefly, the segregation of alleles from heterozygous parents to their affected offspring is tested. The portion of alleles transmitted to the affected offspring compared to the non-transmitted alleles is compared to the ratio expected under random distribution. A significant excess of allele transmission over the expected value is evidence for an association of the respective allele or haplotype with the studied obesity phenotype.

The results of this analysis show that certain alleles of the CNTNAP2 gene are positively associated with obesity and therefore increase the susceptibility to disease. In the tested population, for example, the allele 1 of SNP 156 is correlated with obesity as determined by TDT (p-value=0.000246). In contrast, the allele 2 of SNP156 is significantly under-transmitted to autistic individuals showing that this allele helps protect from the disease. Other SNPs associated with obesity include SNP45, SNP48, SNP49, SNP50, SNP51, SNP53, SNP56, SNP60, SNP63, SNP65, SNP77, SNP94, SNP111, SNP112, SNP141, SNP142, and SNP163 as shown in the examples of the transmission of the alleles to obese patients in Table 3.

Examples of the transmission and non-transmission of the alleles to obese patients are given in Table 3.

TABLE 3

| | | Allele | Allele not | |
|---|---|---|---|---|
| | | transmitted | transmitted | |
| | | to obese | to obese | |
| SNP | Allele | individuals (N) | individuals (N) | p-value |
| SNP45 | 1 | 121 | 92 | 0.01399 |
| SNP45 | 2 | 188 | 217 | 0.01399 |
| SNP48 | 1 | 119 | 154 | 0.03415 |
| SNP48 | 2 | 154 | 119 | 0.03415 |
| SNP49 | 1 | 144 | 107 | 0.019522 |
| SNP49 | 2 | 107 | 144 | 0.019522 |
| SNP50 | 1 | 176 | 138 | 0.031996 |
| SNP50 | 2 | 138 | 176 | 0.031996 |
| SNP51 | 1 | 166 | 194 | 0.02227 |
| SNP51 | 2 | 143 | 115 | 0.02227 |
| SNP53 | 1 | 131 | 167 | 0.037031 |
| SNP53 | 2 | 167 | 131 | 0.037031 |
| SNP56 | 1 | 175 | 208 | 0.007016 |
| SNP56 | 2 | 140 | 107 | 0.007016 |
| SNP60 | 1 | 208 | 166 | 0.029873 |
| SNP60 | 2 | 166 | 208 | 0.029873 |
| SNP63 | 1 | 167 | 208 | 0.03424 |
| SNP63 | 2 | 208 | 167 | 0.03424 |
| SNP65 | 1 | 198 | 160 | 0.044605 |
| SNP65 | 2 | 160 | 198 | 0.044605 |
| SNP77 | 1 | 204 | 178 | 0.03646 |
| SNP77 | 2 | 117 | 143 | 0.03646 |
| SNP94 | 1 | 140 | 109 | 0.049467 |
| SNP94 | 2 | 109 | 140 | 0.049467 |
| SNP111 | 1 | 156 | 110 | 0.004796 |
| SNP111 | 2 | 110 | 156 | 0.004796 |
| SNP112 | 1 | 118 | 87 | 0.030377 |
| SNP112 | 2 | 87 | 118 | 0.030377 |
| SNP141 | 1 | 191 | 152 | 0.035222 |
| SNP141 | 2 | 152 | 191 | 0.035222 |
| SNP142 | 1 | 184 | 139 | 0.012284 |
| SNP142 | 2 | 139 | 184 | 0.012284 |
| SNP156 | 1 | 118 | 68 | 0.000246 |
| SNP156 | 2 | 68 | 118 | 0.000246 |
| SNP163 | 1 | 262 | 240 | 0.02604 |
| SNP163 | 2 | 50 | 72 | 0.02604 |

In addition, haplotypes were constructed for all the SNPs included in Table 3 apart from SNP94 plus SNP11, SNP14, SNP40, SNPSNP47, SNP65, SNP113, SNP116, SNP142 and SNP161 to identify the phase for all SNPs.

The results of this analysis in the tested population showed that certain haplotypes are strongly associated with obesity, while certain haplotypes are preferentially not transmitted to obese patients. An example for a haplotype that is preferentially transmitted to obese patients is the haplotype 1-2-1-1 for SNP50-SNP56-SNP113-SNP156, p=4.946⁻⁰⁶. An example for a haplotype that is preferentially not transmitted to obese patients is the haplotype 2-1-1-2 for SNP50-SNP56-SNP113-SNP156, p=0.0007862.

Examples of haplotypes with preferential transmission and non-transmission to obese patients are given in Table 4.

TABLE 4

| | | Frequency of | Frequency of | |
|---|---|---|---|---|
| SNPs used to | Alleles | haplotype | haplotype not | |
| construct | composing | transmitted to | transmitted to | |
| haplotype | haplotype | obese patients | obese patients | p-value |
| 40-50-156 | 1-1-1 | 0.2631 | 0.1273 | 2.12e−05 |
| 40-50-156 | 1-2-2 | 0.05078 | 0.1181 | 0.001761 |
| 50 113 156 | 1-1-1 | 0.2792 | 0.1342 | 1.312e−05 |
| 50 113 156 | 2-1-2 | 0.05991 | 0.1241 | 0.004561 |
| 50-116-156 | 1-2-1 | 0.1732 | 0.06897 | 7.213e−05 |
| 50-56-113-156 | 1-2-1-1 | 0.2481 | 0.1068 | 4.946e−06 |
| 50-56-113-156 | 2-1-1-2 | 0.03738 | 0.1069 | 0.0007862 |
| 40-50-56-156 | 1-1-2-1 | 0.2211 | 0.0916 | 7.59e−06 |
| 40-50-56-156 | 1-2-1-2 | 0.0341 | 0.09947 | 0.001165 |
| 40-56-113-156 | 1-1-1-2 | 0.02471 | 0.07988 | 0.002167 |
| 40-56-113-156 | 1-2-1-1 | 0.2581 | 0.1238 | 4.269e−05 |
| 40-50-113-156 | 1-1-1-1 | 0.2135 | 0.08298 | 8.213e−06 |
| 45-48-156 | 1-2-1 | 0.2398 | 0.1192 | 0.000123 |
| 45-48-156 | 2-1-2 | 0.0832 | 0.1322 | 0.02454 |
| 45-14-156 | 1-1-1 | 0.1341 | 0.05972 | 0.001942 |
| 45-14-156 | 1-2-1 | 0.2103 | 0.1429 | 0.02729 |
| 45-14-156 | 1-2-2 | 0.02364 | 0.06296 | 0.01976 |
| 45-14-156 | 2-1-2 | 0.02232 | 0.05554 | 0.02821 |
| 45-14-156 | 1-1-1 | 0.1782 | 0.08875 | 0.0007682 |
| 45-14-156 | 1-2-2 | 0.01635 | 0.06292 | 0.003773 |
| 45-14-156 | 2-1-2 | 0.02617 | 0.06711 | 0.01712 |
| 47-48-156 | 1-1-2 | 0.0903 | 0.1474 | 0.01483 |
| 47-48-156 | 2-2-1 | 0.2511 | 0.1312 | 0.0002089 |
| 47-11-156 | 1-2-2 | 6.54e−11 | 0.08162 | 2.907e−07 |
| 47-11-156 | 2-1-1 | 0.2521 | 0.181 | 0.006931 |
| 48-65-156 | 1-1-2 | 0.04111 | 0.1012 | 0.004426 |
| 48-65-156 | 2-2-1 | 0.174 | 0.1014 | 0.009455 |
| 48-11-156 | 1-2-2 | 0.004645 | 0.08581 | 4.385e−07 |
| 48-11-156 | 2-1-1 | 0.2227 | 0.1416 | 0.005923 |
| 49-141-156 | 1-1-1 | 0.1592 | 0.0509 | 0.0001743 |
| 49-141-156 | 2-1-2 | 0.02592 | 0.05667 | 0.04417 |
| 50-51-156 | 1-2-1 | 0.3344 | 0.1934 | 0.0001126 |
| 50-51-156 | 2-1-2 | 0.0788 | 0.1529 | 0.002454 |
| 50-60-111 | 1-1-1 | 0.1549 | 0.08485 | 0.00394 |
| 50-60-111 | 2-1-2 | 0.05278 | 0.1177 | 0.007073 |
| 50-60-156 | 1-1-1 | 0.1799 | 0.0997 | 0.002196 |
| 50-60-156 | 1-2-1 | 0.163 | 0.1067 | 0.04082 |
| 50-60-156 | 2-1-2 | 0.05528 | 0.1003 | 0.02889 |
| 50-60-156 | 2-2-1 | 0.2105 | 0.2953 | 0.01913 |
| 50-63-156 | 1-1-1 | 0.1877 | 0.1 | 0.002533 |
| 50-63-156 | 1-2-1 | 0.1581 | 0.1081 | 0.03409 |
| 50-63-156 | 2-1-1 | 0.2673 | 0.3598 | 0.02274 |
| 50-63-156 | 2-2-2 | 0.04493 | 0.08649 | 0.02845 |
| 50-65-156 | 1-1-1 | 0.1605 | 0.1065 | 0.02477 |
| 50-65-156 | 1-2-1 | 0.1861 | 0.09773 | 0.002379 |
| 50-65-156 | 2-1-2 | 0.03714 | 0.08847 | 0.00934 |
| 50-65-156 | 2-2-1 | 0.2738 | 0.3678 | 0.02505 |
| 50-112-141 | 1-1-1 | 0.125 | 0.06503 | 0.01111 |
| 50-112-141 | 2-2-1 | 0.01719 | 0.08347 | 0.0005085 |
| 50-141-156 | 1-1-1 | 0.142 | 0.05543 | 0.0004759 |
| 50-141-156 | 1-2-1 | 0.202 | 0.153 | 0.05193 |
| 50-141-156 | 2-1-2 | 0.02591 | 0.057 | 0.03197 |
| 50-142-156 | 1-1-1 | 0.187 | 0.07765 | 5.547e−05 |
| 50-142-156 | 2-1-2 | 0.03671 | 0.0747 | 0.03206 |
| 50-156-161 | 1-1-1 | 0.1628 | 0.0897 | 0.005409 |
| 50-156-161 | 1-1-2 | 0.1796 | 0.1166 | 0.01194 |
| 50-156-161 | 2-2-2 | 0.06924 | 0.1473 | 0.001339 |
| 50-156-163 | 1-1-1 | 0.3015 | 0.1645 | 5.931e−05 |
| 50-156-163 | 1-2-2 | 0.01869 | 0.02398 | 0.02484 |
| 50-156-163 | 2-2-2 | 0.02621 | 0.06409 | 0.01847 |
| 53-60-156 | 1-1-1 | 0.3509 | 0.2698 | 0.03822 |
| 53-60-156 | 1-2-1 | 0.2179 | 0.3078 | 0.01797 |
| 53-60-156 | 2-2-1 | 0.1618 | 0.09989 | 0.03112 |
| 53-111-156 | 1-2-2 | 0.006907 | 0.0784 | 5.113e−06 |
| 53-111-156 | 2-1-1 | 0.2199 | 0.1587 | 0.03123 |
| 53-112-156 | 1-2-2 | 0.01046 | 0.05976 | 0.0004787 |
| 53-112-156 | 2-1-1 | 0.2304 | 0.1548 | 0.0132 |
| 60-77-111 | 1-1-1 | 0.2695 | 0.1949 | 0.02817 |
| 60-77-111 | 1-1-2 | 0.04658 | 0.1062 | 0.02026 |
| 60-77-111 | 2-2-1 | 0.09236 | 0.1544 | 0.02134 |
| 60-77-111 | 2-2-2 | 0.03902 | 0.09576 | 0.00513 |
| 60-77-142 | 1-1-1 | 0.1677 | 0.09388 | 0.007573 |
| 60-77-142 | 2-2-1 | 0.05143 | 0.08728 | 0.09579 |
| 60-77-142 | 2-2-2 | 0.07952 | 0.1637 | 0.001891 |
| 60-111-156 | 1-1-1 | 0.3934 | 0.2572 | 0.0003834 |
| 60-111-156 | 1-2-2 | 0.004562 | 0.05028 | 0.0004276 |
| 60-111-156 | 2-2-2 | 0.006349 | 0.03684 | 0.00462 |
| 63-77-111 | 1-2-2 | 0.04488 | 0.1126 | 0.002868 |
| 63-77-111 | 2-1-2 | 0.03732 | 0.09388 | 0.01354 |
| 63-111-156 | 1-2-2 | 0.005687 | 0.04263 | 0.01254 |
| 63-111-156 | 2-1-1 | 0.3211 | 0.227 | 0.006942 |
| 63-111-156 | 2-2-2 | 0.005947 | 0.04458 | 3.394e−05 |
| 77-111-156 | 1-1-1 | 0.4369 | 0.3339 | 0.005773 |
| 77-111-156 | 1-2-2 | 0.008825 | 0.03817 | 0.01577 |
| 77-111-156 | 2-2-2 | 0.004723 | 0.05016 | 0.0002808 |
| 111-142-156 | 1-1-1 | 0.3236 | 0.2381 | 0.01006 |
| 111-142-156 | 2-1-2 | 0.007099 | 0.04145 | 0.01948 |
| 111-142-156 | 2-2-2 | 0.006951 | 0.04059 | 0.0008523 |
| 111-156-161 | 1-2-1 | 0.02353 | 0.01563 | 0.05112 |
| 111-156-161 | 2-2-1 | 0.01273 | 0.008456 | 0.05269 |
| 111-156-161 | 2-2-2 | 0.007709 | 0.07174 | 2.86e−05 |
| 111-156-163 | 1-1-1 | 0.5671 | 0.4723 | 0.01049 |
| 111-156-163 | 2-2-1 | 0.005063 | 0.05258 | 0.0006018 |
| 111-156-163 | 2-2-2 | 0.01374 | 0.02757 | 0.002219 |

### REFERENCES

Baumann M H, Ayestas M A, Dersch C M et al. (2000) Serotonin transporters, serotonin release, and the mechanism of fenfluramine neurotoxicity. Ann N Y Acad Sci 914:172-86
Chagnon Y C, Rankinen T, Snyder E E et al. (2003) The human obesity gene map: The 2002 update. Obes Res. 11(3):313-367.
Hebebrand J, Hescker H, Himmelmann G W, Schäfer H, Remschmidt H (1994) Percentiles for the body mass index based on data of the German national nutrition survey and a review of relevant factors with an influence on body weight. Aktuelle Ernaehrungsmedizin 19: 259-265.
Hebebrand J, Himmelmann G W, Hescker H, Schäfer H, Remschmidt H (1996) Use of percentiles for the body mass index in anorexia nervosa: diagnostic, epidemiological and therapeutic considerations. Int J Eat Dis 19: 359-369.
Hebebrand J, Hinney A, Roth H et al. (1998) Genetische Aspekte der Adipositas. In:J Wechsler (ed) Adipositas. Ex Libris Roche. Blackwell Verlag: 105-118.
Hinney A, Schmidt A, Nottebom K et al. (1999) Several mutations in the melanocortin-4 receptor gene including a nonsense and a frameshift mutation associated with dominantly inherited obesity in humans. J. Clin Endocrinol Metab. Apr; 84(4): 1483-1486.
Hu S, Wang S, Gibson J et al (1998) Inhibition of delayed rectifier K+ channels by dexfenfluramine (Redux). J Pharmacol Exp Ther. 287(2):480-6.
Hugot J P, Chamaillard M, Zouali H et al. (2001) Association of NOD2 leucine-rich repeat variants with susceptibility to Crohn's disease. Nature 411 (6837):599-603.
Huszar D, Lynch C A, Fairchild-Huntress V et al. (1997) Targeted disruption of the melanocortin-4 receptor results in obesity in mice. Cell 88:131-141.
Lander E and Kruglyak L (1995) Genetic dissection of complex traits: guidelines for interpreting and reporting linkage results. Nat Genet, 11 (3):241-247.
Lesage S, Zouali H, Cezard Jp et al. (2002) CARD15/NOD2 mutational analysis and genotype-phenotype correlation in 612 patients with inflammatory bowel disease. Am J Hum Genet. 70(4):845-857.
MacDonald P E and Wheeler M B (2003) Voltage-ependent K+ channels in pancreatic beta cells: Role, regulation and potential as therapeutic target. Diabetologia 46:1046-1062.
McDaniel S S, Platoshyn 0, Yu Y et al. (2001) Anorexic effect of K+ channel blockade in mesenteric arterial smooth muscle and intestinal epithelial cells. J Appl Physiol. 91 (5):2322-2333.
Michelakis E D, Weir E K, Nelson D P et al. (1999) Dexfenfluramine elevates systemic blood pressure by inhibiting potassium currents in vascular smooth muscle cells. J Pharmacol Exp Ther. 291(3):1143-9.
Ogura Y, Bonen D K, Inohara N (2001) A framshift mutation in NOD2 associated with susceptibility to Crohn's disease. Nature 411(6837):603-606.
Poliak S, Gollan L, Martinez R et al. (1999) Caspr2, a new member of the neurexin superfamily, is localized at the juxtaparanodes of myelinated axons and associates with K+ channels. Neuron 24(4):1037-1047.
Rioux J D, Daly M J, Silverberg M S et al. (2001) Genetic variation in the 5q31 cytokine gene cluster confers susceptibility to Crohn disease. Nat Genet. 29(2): 223-228.
Rioux J D, Silverberg M S, Daly M J (2000) Genomewide search in Canadian families with inflammatory bowel disease reveals two novel susceptibility loci. Am J Hum Genet. 66(6):1863-1870.
Schneider R (1996) Relevanz und Kosten der Adipositas in Deutschland. Ernährungs-Umschau 43:369-374.
Vaisse C, Clement K, Guy-Grand B et al. (1998) A frameshift mutation in human MC4R is associated with a dominant form of obesity. Nat Genet 20:113-114.
Wang J, Juhaszova M, Rubin L J et al. (1997) Hypoxia inhibits gene expression of voltage-gated K+ channel alpha subunits in pulmonary artery smooth muscle cells. J Clin Invest. 100(9):2347-53.
WHO (1998) Preventing and managing the global epidemic. WHO, Geneva
Yeo G S, Farooqi I S, Aminian S et al. (1998) A frameshift mutation in MC4R associated with dominantly inherited human obesity. Nat Genet 20:111-112.
Wolf A M and Colditz G A (1996) Social and economic effects of body weight in the United States. Am J Clin Nutr 63(3 Suppl):466S469S.
Xu J, Koni P A, Wang P et al. (2003) The voltage-gated potassium channel Kv1.3 regulates energy homeostasis and body weight. Hum Mol Genet. 12(5):551-9.
Zhang Y, Proenca R, Maffei M, Barone M, Leopold L, Friedman J M. (1994) Positional cloning of the mouse obese gene and its human homologue. Nature 372: 425-432.

1. A method of detecting the presence of or predisposition to obesity in a subject, the method comprising (i) providing a sample from the subject and (ii) detecting the presence of an alteration in the CNTNAP2 gene locus in said sample.

2-5. (canceled)

6. The method of claim 1, wherein the presence of an alteration in the CNTNAP2 genes locus is detected by sequencing, selective hybridisation and/or selective amplification.

7. The method of claim 1, wherein said alteration is one or several SNP(s) or a haplotype of SNPs associated with obesity.

8. The method of claim 7, wherein said haplotype associated with obesity comprises several SNPs selected in the group consisting of SNP11, SNP14, SNP40, SNP45, SNP47, SNP48, SNP49, SNP50, SNP51, SNP53, SNP56, SNP60, SNP63, SNP65, SNP77, SNP94, SNP111, SNP112, SNP113, SNP116, SNP141, SNP142, SNP156, SNP161, and SNP163.

9. The method of claim 7, wherein said SNP associated with obesity is selected from the group consisting of SNP45, SNP48, SNP49, SNP50, SNP51, SNP53, SNP56, SNP60, SNP63, SNP65, SNP77, SNP94, SNP111, SNP112, SNP141, SNP142, SNP156, SNP161, and SNP163.

10. The method of claim 9, wherein said SNP is associated with a protection against obesity.

11. The method of claim 10, wherein said SNP associated with protection against obesity is SNP156.

12-19. (canceled)

20. A method of detecting the presence of or predisposition to diabetes in a subject, the method comprising (i) providing a sample from the subject and (ii) detecting the presence of an alteration in the CNTNAP2 gene locus in said sample.

21. The method of claim 20, wherein the presence of an alteration in the CNTNAP2 genes locus is detected by sequencing, selective hybridisation, or selective amplification.

22. The method of claim 20, wherein said alteration is one or several SNPs or a haplotype of SNPs associated with diabetes.

23. The method of claim 22, wherein said haplotype associated with diabetes comprises several SNPs selected in the group consisting of SNP11, SNP14, SNP40, SNP45, SNP47, SNP48, SNP49, SNP50, SNP51, SNP53, SNP56, SNP60, SNP63, SNP65, SNP77, SNP94, SNP111, SNP112, SNP113, SNP116, SNP141, SNP142, SNP156, SNP161, and SNP163.

24. The method of claim 22, wherein said SNP is associated with diabetes is selected from the group consisting of SNP45, SNP48, SNP49, SNP50, SNP51, SNP53, SNP56, SNP60, SNP63, SNP65, SNP77, SNP94, SNP111, SNP112, SNP141, SNP142, SNP156, SNP161, and SNP163.

25. The method of claim 24, wherein said SNP is associated with protection against diabetes.

26. The method of claim 25, wherein said SNP associated with protection against obesity is SNP156.

27. A method for preventing obesity in a subject, comprising detecting the presence of an alteration in the CNTNAP2 gene locus in a sample from the subject, the presence of said alteration being indicative of the predisposition to obesity; and, administering a prophylactic treatment against obesity.

28. A method for preventing diabetes in a subject, comprising detecting the presence of an alteration in the CNTNAP2 gene locus in a sample from the subject, the presence of said alteration being indicative of the predisposition to diabetes; and, administering a prophylactic treatment against diabetes.
